SEQUENCE LISTING (16 sequences)

SEQ ID NO: 1 - DNA, 79 bp, artificial sequence
  Synthetic
  misc_feature (31)..(44): n is a, c, g, or t
  x (45)..(48): x may be any one of a t c g
ctctttccctacacgacgctcttccgatctnnnnnnnnnnnnnnrrrrwgtgttcactagcaaycctcaaacagacacc

SEQ ID NO: 2 - DNA, 49 bp, artificial sequence
  Synthetic
aatgatacggcgaccaccgagatctacactctttccctacacgacgctc

SEQ ID NO: 3 - DNA, 76 bp, artificial sequence
  Synthetic
  misc_feature (35)..(39): n is a, c, g, or t
gtgactggagttcagacgtgtgctcttccgatctnnnnnrrrratccacgttcaccttgcyccacagggcagtaac

SEQ ID NO: 4 - DNA, 76 bp, artificial sequence
  Synthetic
gtgactggagttcagacgtgtgctcttccgatctagtgtaaaaatccacgttcaccttgcyccacagggcagtaac

SEQ ID NO: 5 - DNA, 27 bp, artificial sequence
  Synthetic
aatgatacggcgaccaccgagatctac

SEQ ID NO: 6 - DNA, 25 bp, artificial sequence
  Synthetic
gtgactggagttcagacgtgtgctc

SEQ ID NO: 7 - DNA, 53 bp, artificial sequence
  Synthetic
caagcagaagacggcatacgagatrrrrrrgtgactggagttcagacgtgtgc

SEQ ID NO: 8 - DNA, 80 bp, artificial sequence
  Synthetic
tacacgacgctcttccgatctatggtgcayctgactcctgaggagaagtctgccgttaagatcggaagagcacacgtctg

SEQ ID NO: 9 - DNA, 80 bp, artificial sequence
  Synthetic
tacacgacgctcttccgatctatggtgcayctgactttatgttagaagtctgccgttaagatcggaagagcacacgtctg

SEQ ID NO: 10 - DNA, 41 bp, artificial sequence
  Synthetic
acactctttccctacacgacgctcttccgatctatggtgca

SEQ ID NO: 11 - DNA, 42 bp, artificial sequence
  Synthetic
gtgactggagttcagacgtgtgctcttccgatcttaacggca

SEQ ID NO: 12 - DNA, 66 bp, artificial sequence
  Synthetic
aatgatacggcgaccaccgagatctacactctttccctacacgacgctcttccgatctatggtgca

SEQ ID NO: 13 - DNA, 53 bp, artificial sequence; identical to SEQ ID NO: 7
  Synthetic

SEQ ID NO: 14 - DNA, 114 bp, human
actgtgttcactagcaacctcaaacagacaccatggtgcacctgactcctgaggagaagtctgccgttactgccctgtggggcaaggtgaacgtggatgaagttggtggtgagg

SEQ ID NO: 15 - DNA, 114 bp, human
acagtgttcactagcaacctcaaacagacaccatggtgcatctgactcctgaggagaagactgctgtcaatgccctgtggggcaaagtgaacgtggatgcagttggtggtgagg

SEQ ID NO: 16 - protein, 18 aa, human
MVHLTPEEKSAVTALWGK

(19) United States
(12) Patent Application Publication
(10) Pub. No.: US 2024/0254550 A1
(43) Pub. Date: Aug. 1, 2024

(54) A METHOD OF IDENTIFYING ULTRA-RARE GENETIC VARIANTS

(71) Applicant: CARMEL - HAIFA UNIVERSITY ECONOMIC CORPORATION LTD, Haifa (IL)

(72) Inventors: Adi LIVNAT, Haifa (IL); Daniel MELAMED, Kibbutz Ein Hamifratz (IL)

(21) Appl. No.: 18/290,552

(22) PCT Filed: May 12, 2022
(86) PCT No.: PCT/IL2022/050502

§ 371 Date: Nov. 14, 2023

Related U.S. Application Data
(60) Provisional application No. 63/188,627, filed on May 14, 2021.

(51) Int. Cl. C12Q 1/6869; C12N 15/10; C12Q 1/6858

(52) U.S. Cl. CPC C12Q 1/6869; C12N 15/1065; C12Q 1/6858

(57) ABSTRACT

The invention concerns methods of identifying genetic variants in a DNA sample comprising a first step of removing wild-type DNA sequences thereby enriching the sample with DNA sequences containing the genetic variant, while calculating an enrichment factor, and a second step of determining the number of genetic variants in said DNA sample.

### TECHNOLOGICAL FIELD

The present disclosure is in the field of genetic variants detection, especially the detection of ultra-rare variants in large cell populations or in cell free DNA.

### BACKGROUND

While Next Generation Sequencing (NGS) technology has tremendously improved the cost and scale of DNA sequencing, the detection of extremely rare genetic variants is still a major challenge. This unresolved problem is due to both DNA polymerase errors that are introduced during sample preparation and sequencing errors made by the NGS machinery. DNA polymerase error rates range between ˜1×10⁻⁴ per base for Taq polymerase to ˜1×10⁻⁶ per base for various high-fidelity DNA polymerases (Hestand MS, et al (2016) Mutat. Res. 784:39-45; Lee DF, et al (2016) Nucleic Acids Res. 44(13): e118-e118; Potapov V and Ong JL (2017) PloS One 12(1)), and error rates of the commonly used NGS platforms range between 10⁻²-10⁻³ per sequenced base (Fox EJ, et al (2014) Next Gener. Seq Appl 1), with some computational efforts being able to enhance sequencing accuracy 10-100-fold (Ma X, et al. (2019) Genome Biol 20(1):50; Cibulskis K, et al. (2013) Nat Biotechnol. 31(3):213). These abilities allow for the detection of some sub-populations of sequences within a highly homogenous sample. However, given that the average per-base point mutation rate across the human genome is ˜10^{-8,} entailing on average ˜10⁻⁸ wild-type copies per mutant, the rates above would lead to numerous false positives per one true base mutation. Thus, detecting a single instance of a particular de novo mutation in a particular gene has been practically impossible so far. Moreover, even in the absence of errors, obtaining enough reads of such a mutation by sequencing alone would have entailed an exorbitant sequencing cost.

In recent years, a few experimental approaches have been developed that substantially reduce the noise generated by both DNA polymerase and NGS errors (for example, Jee J, et al. (2016) Nature 534(7609):693; U.S. Pat. No. 10,513,732; Kinde I, et al (2011) Proc. Natl. Acad. Sci. USA 108(23):9530-9535). One key idea has been to attach a unique molecular tag or “barcode” to each DNA fragment at the first PCR cycle during the amplification step. After library preparation and standard NGS sequencing, reads that share the same barcode are recognized as having been derived from the same original molecule. Since those reads should be identical, the differences between them are considered as errors introduced during PCR and/or NGS sequencing and are filtered out at the analysis stage (Kinde I, et al (2011) Supra). This filtration step removes many of the DNA polymerase and NGS errors that occur after barcode attachment.

Importantly, however, the standard way by which the barcode has been added to the target DNA is by being included as a part of a target-specific primer that is extended by a single elongation reaction, generating a sequence subsequently to be amplified using an external pair of primers. A major disadvantage of this standard method is that any replication error introduced by the DNA polymerase during the critical, initial copying of the original DNA molecule is transferred to all downstream copies during the PCR reaction and cannot be filtered out by the regular barcoding-and-consensus-sequencing approach.

To overcome this problem, a few methods have been developed, e.g., Duplex Sequencing (DS) (Schmitt MW, et al. (2012) Proc. Natl. Acad. Sci. USA 109(36):14508-14513) However, while ideal for genomic regions at the 1 Mb scale, used on smaller regions, would entail extremely low yields, which in turn would make it impossible to reduce the size of the region of interest (ROI) and increase the sequencing depth in a manner nearly cost-effective enough as to focus on a particular mutation of interest.

An alternative method that avoids errors in the first copying step is Maximum Depth Sequencing (MDS) (Jee J, et al. (2016) Supra). In contrast to DS, MDS can potentially minimize the ROI to a single base in the genome. Since MDS recovers the sequence information from only one of the two DNA strands, however, it cannot correct certain kinds of error due to DNA damage or base misincorporation by the cellular DNA polymerase that affect the target DNA strand, in contrast to DS. Yet, eliminating these highly frequent, known types of errors from the mutation rate calculation resulted in a tested MDS error rate of about 1×10⁻⁷ while using Phusion DNA polymerase, and a suggested theoretical error rate of less than 5×10⁻⁸ if Q5 DNA polymerase is used (Jee J, et al. (2016) Supra).

While both DS and MDS reach extraordinary levels of precision, their error rates and sequence coverage still pose serious difficulties in detecting specific mutations occurring at or near the human genome-wide average mutation rate. It should be noted that natural mutation variants constitute a tiny fraction of the target DNA molecules, while most of the target DNA consists of a common, non-mutated sequence, which is referred to as the “wild-type” sequence. This fact has two negative consequences. First, since each wild-type molecule is one that can be mistakenly read as a mutation, the wild type is a ubiquitous source of false positives. Second, since the goal is to detect mutations, most of the sequencing capacity and costs are devoted to sequence copies that are of little interest. Therefore, there is a clear need to develop a sequencing method that can overcome these limitations.

### GENERAL DESCRIPTION

In one aspect, the present invention provides a method of identifying genetic variants in DNA; said method comprising:
a. Providing a sample of isolated DNA, wherein said DNA comprises wild-type DNA sequences and optionally one or more DNA sequences containing a genetic variant in one or more regions of interest (ROIs);
b. Removing said wild-type DNA sequences from said sample, thereby enriching the sample with DNA sequences containing the genetic variant;
c. Determining the number of the wild-type sequences that were removed by calculating an enrichment factor (E); and
d. Determining the number of genetic variants in said DNA sample.

In one embodiment, said step (b) of removing said wild-type DNA sequences from said sample comprises subjecting said DNA to a first cutting agent and optionally to a second cutting agent, wherein the recognition site for said first cutting agent is within the ROI, and wherein the recognition site for said second cutting agent is in proximity to the ROI, whereby wildtype ROI is cut by the first cutting agent and an ROI which comprises a genetic variation is not cut by said first cutting agent.

In one embodiment, said step (d) of determining the number of genetic variants in said DNA is performed by a sequencing-based method.

In a specific embodiment said sequencing-method is a barcoding-based sequencing method.

In one embodiment, said barcoding-based sequencing method comprises:
i. Attaching a Primary barcode to said ROI products thereby obtaining barcoded ROI products;
ii. Linearly amplifying said barcoded ROI products thereby obtaining amplified barcoded ROI products;
iii. Performing a polymerase chain reaction (PCR) with the amplified barcoded ROI product of step (ii) using at least two primers for next generation sequencing; and
iv. Sequencing the amplified product, thereby obtaining sequencing data; and
v. Analyzing the data obtained in step (iv) to determine the number of genetic variants in said DNA.

In another embodiment, said step (d) of determining the number of genetic variants in said DNA is performed by a polymerase chain reaction (PCR).

In a specific embodiment said PCR is quantitative PCR or digital droplet PCR.

In one embodiment, said genetic variants are ultra-rare genetic variants such as one or more de novo mutations.

In one embodiment, said one or more de novo mutation is a specific predefined de novo mutation.

In one embodiment, said sample comprises a cell population comprising between about ≤10,000 cells and about 1×10⁹ cells.

In one embodiment, said method identifies genetic variants with a maximal error rate of 1 per 400 million bases.

In one embodiment, said step (b) of removing said wild-type DNA sequences from said sample, thereby enriching the sample with DNA sequences containing the genetic variant results in obtaining a population of isolated ROI products comprising one or more target mutations, wherein wild-type ROI sequences were substantially removed from said population.

In one embodiment, said step (i) of attaching a Primary barcode to said ROI products comprises forming a mixture comprising the cut DNA and an oligonucleotide, wherein said oligonucleotide comprises a primary barcode, a primer sequence, and optionally a sample-identifier sequence, and wherein said oligonucleotide anneals to the sequence between the recognition site of the first cutting agent and the recognition site of the second cutting agent.

In one embodiment, said primer sequence is an Illumina P5-primer sequence.

In one embodiment, said step (i) further comprises attaching a base modification that blocks DNA polymerase from extending said oligonucleotide and optionally further comprises planting a control single-base insertion.

In one embodiment, said base modification that blocks DNA polymerase from extending said oligonucleotide is a 3′ inverted-dT (3′inv-dT).

In one embodiment, said step (ii) of linearly amplifying said barcoded ROI products comprises performing one or more cycles of linear amplification using an oligonucleotide that anneals to the primer sequence of the target DNA strand, thereby obtaining copies in an amount that is equal or less than the number of linear cycles of each barcoded target molecule.

In one embodiment, said step (ii) comprises performing between 2 cycles and 20 cycles of linear amplification.

In one embodiment, the method further comprises subjecting said amplified barcoded ROI products obtained in step (ii) to degradation by a 5′-exonuclease enzyme.

As used herein the term “5′-exonuclease enzyme” refers to any enzyme that is capable of cleaving nucleotides from the 5′ end (exo) of a polynucleotide chain.

In one embodiment, the DNA is larger than 10 million base pairs.

In one embodiment, said method further comprises adding an adapter sequence comprising one or more base modifications that protect a barcoded-ROI copy from 5′ exonuclease degradation at the 5′ edge (5′PS) of each barcoded-ROI copy.

In one embodiment, said adapter sequence is an Illumina adapter sequence.

In one embodiment, said adapter sequence comprises 5 base modifications.

In one embodiment, said base modifications are phosphorothioate bonds.

In one embodiment, the method further comprises attaching a secondary barcode after said amplified barcoded ROI products were subjected to degradation by a 5′-exonuclease enzyme, thereby obtaining a double-stranded barcoded ROI.

In one embodiment, the method further comprises degrading said amplified barcoded ROI using a 3′-exonuclease prior to sequencing the amplified product.

In one embodiment, said step (c) of determining the number of the wild-type sequences that were removed by calculating an enrichment factor (E) is performed in parallel with step (b) of removing said wild-type DNA sequences from said sample.

In one embodiment, said step of determining the number of the target wild-type sequences that were removed by calculating an enrichment factor (E) comprises the steps of:
a. Providing mock DNA comprising copies of an artificial sequence that is resistant (R) to cutting by the first cutting agent;
b. Subjecting a fraction of said mock DNA to a first cutting agent and optionally to a second cutting agent, wherein the recognition site for said first cutting agent is within the ROI, and wherein the recognition site for said second cutting agent is in proximity to the ROI, whereby wildtype ROI is cut by the first cutting agent and an ROI which comprises a genetic variation is not cut by said first cutting agent; and further subjecting the fraction of said mock DNA to steps (a) to (d) of claim 5, thereby obtaining sequence data for mock DNA subjected to cutting (referred to herein as Group III);
c. In parallel, subjecting another fraction of said mock DNA only to said second cutting agent; and
further subjecting the fraction of said mock DNA to steps (b) to (d) of claim 5, thereby obtaining sequence data for mock DNA that was not subjected to said first cutting agent (referred to herein as Group IV);
d. Providing isolated DNA comprising a region of interest (ROI) in accordance with claim 1 step (a);
e. Subjecting a fraction of said DNA to a first cutting agent and optionally to a second cutting agent, wherein the recognition site for said first cutting agent is within the ROI, and wherein the recognition site for said second cutting agent is in proximity to the ROI, whereby wildtype ROI is cut by the first cutting agent and an ROI which comprises a genetic variation is not cut by said first cutting agent; and
further subjecting the fraction of said DNA to steps (i) to (iv) described above, thereby obtaining sequence data for DNA subjected to cutting (referred to herein as Group I);
f. In parallel, subjecting another fraction of said DNA only to said second cutting agent; and further subjecting the fraction of said DNA to steps (ii) to (iv) described above, thereby obtaining sequence data for DNA that was not subjected to said first cutting agent (referred to herein as Group II); and
g. comparing the ratios between the numbers of DNA molecules that are sensitive (S) and resistant (R) to the cutting agent between the mixture that was subjected to cutting the wild-type and the mixture that was not subjected to cutting the wild-type,
thereby determining the number of the target wild-type sequences that were removed.

In one embodiment, the enrichment factor (E) is determined by the formula

$$E = \frac{Rfe \times Sfc \times VSe \times VRc}{Sfe \times Rfc \times VRe \times VSc}$$

Wherein

R^{f}ₑ is the number of artificial, resistant molecules measured in Group III,

S^{f}_{c} is the number of sensitive (i.e., wildtype) molecules measured in Group II;

V_{Se} is the volume taken from the DNA tube for Group I;

V_{Rc} is the volume taken from the mock DNA tube for Group IV;

S^{f}ₑ is the number of sensitive (i.e., wildtype) molecules measured in Group I;

R^{f}_{c} is the number of artificial, resistant molecules measured in Group IV;

V_{Re} is the volume taken from the mock DNA tube for treatment in Group III; and

V_{Sc} is the volume taken from the DNA tube for Group II.

In one embodiment, the step of analyzing the data obtained to determine the number of genetic variants in said DNA comprises using combined threshold criteria, wherein said criteria comprise:
a. primary-barcode family size,
b. within-family mutation frequency cutoff, and
c. association of at least two secondary barcodes with each base.

In one embodiment, said combined threshold criteria comprise:
a. primary-barcode families with at least three reads,
b. a minimal within-family mutation frequency cutoff of 70%, and
c. the association of at least two secondary barcodes with each base.

In one embodiment, the step of analyzing the data obtained to determine the number of genetic variants in said DNA comprises the algorithm as shown in FIG. 8.

In one embodiment, said first cutting agent and said second cutting agent is an enzyme.

In one embodiment, said enzyme is an endonuclease or a restriction enzyme.

In one embodiment, said first cutting agent is an enzyme that cleaves at the ROI to produce digested DNA.

In one embodiment, said first cutting agent and said second cutting agent are the same.

In one embodiment, said first and/or second cutting agent is a CRISPR-Cas9 agent.

In one embodiment, said DNA is genomic DNA or synthetic DNA.

In one embodiment, said sample of isolated DNA is a biological sample and wherein said biological sample is selected from a group consisting of semen, amniotic fluid, blood, cerebrospinal fluid, ascitic fluid, saliva, urine, bronchoalveolar lavage fluid, or nasal lavage fluid, or a tissue biopsy.

In one embodiment, said sample of isolated DNA is a non-biological sample and wherein said non-biological sample is selected from a group consisting of a soil sample, a sewer sample, water sample, and a sample taken from a solid surface.

In another aspect, the present invention provides a method of diagnosis or prognosis of a disease or disorder or a method of determining the likelihood of developing a disease or disorder or defining the progress of a disease or disorder comprising identifying at least one genetic variant in accordance with the methods of the invention.

In one embodiment, said disease or disorder is a proliferative disease or cancer.

In one embodiment, said cancer is lymphoma or leukemia.

In one embodiment, said disorder is a genetic disorder.

In one embodiment, said genetic disorder is a fetal genetic disorder.

In one embodiment, said sample is a maternal blood sample or amniotic fluid.

In one embodiment, said disease is a viral disease or a bacterial disease.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A-FIG. 1H is a schematic illustration of the MEMDS method. FIG. 1A illustrates Step 1: Enzymatic digestion of Mutant and Wild type genomic DNA. RE-1 and RE-2 represent Restriction Enzyme-1 and 2 respectively. The wild-type sequence is RE-1 sensitive and therefore is being digested while the mutant sequence is RE-1 resistant and therefore remains intact. Lightning arrows indicate the illustrative digestion sites of the restriction enzymes. FIG. 1B illustrates Step 2: primary barcode attachment to the Mutant and Wild type DNA strands. A fill-in reaction is promoted by oligo A, which anneals the target DNA strand (gray strand) a sample-identifier sequence (ID-1), a primary-barcode sequence (BC1) and an Illumina-P5 sequence. 3′inv-dT—3′ inverted-dT; ins—control base-insertion. FIG. 1C illustrates Step 3: linear amplification of the Mutant and Wild type DNA strands using oligo B which adds an adapter sequence with 5 phosphorothioate bonds at the 5′ edge (PS). This linear amplification reaction results in 15 or less copies of each barcoded target molecule (N≤15). A circle represents a polymerase error. FIG. 1D illustrates Step 4: 5′ exonuclease treatment. FIG. 1E illustrates Step 5: Secondary barcode attachment. An extension reaction with oligo C is carried out to add to each target molecule an additional sample identifier sequence (ID-2), a unique secondary-barcode sequence (BC2), and an Illumina-P7 sequence (P7). For each target molecule, this step results in 15 or less copies. The wild type is not labeled by this oligo. A circle represents a polymerase error. FIG. 1F illustrates Step 6: 3′ exonuclease treatment. FIG. 1G illustrates Step 7: PCR amplification. FIG. 1H illustrates Step 8: Sequence analysis.

FIG. 2 is a schematic illustration of the MEMDS experimental design to calculate the RE-1-enrichment factor and the number of target DNA molecules digested by RE-1. Abbreviations: [S]—Concentration of genomic DNA (mostly RE-1-sensitive) in source tube; [R]—Concentration of artificial DNA (RE-1-resistant) in source tube; V_{Se}—Volume taken from “S” source tube to the RE-1-treated sample; V_{Sc}—Volume taken from “S” source tube to the RE-1-untreated sample; V_{Re}—Volume taken from “R” source tube to the RE-1-treated sample; V_{Rc}—Volume taken from “R” source tube to the RE-1-untreated sample; Lₑ—Amount of “S” and “R” molecules lost during the MEMDS procedure from the RE-1-treated sample; L_{c}—Amount of “S” and “R” molecules lost during the MEMDS procedure from the RE-1-untreated sample; S^{f}ₑ—Number of RE-1-sensitive variants in the RE-1-treated sample; R^{f}ₑ—Number of RE-1-resistant variants in the RE-1-treated sample; S^{f}_{c}—Number of RE-1-sensitive variants in the RE-1-untreated sample; R^{f}_{c}—Number of RE-1-resistant variants in the RE-1-untreated sample; E—RE-1 enrichment factor; W—Number of RE-1 screened wild-type ROIs.

FIG. 3 is a schematic illustration of HBB and HBD sequence features. The double-stranded 114 bp DNA segments from the first exon of HBB (upper sequences; the sense strand is denoted as SEQ ID NO: 14) and the homologous region of HBD (lower sequences; the sense strand is denoted as SEQ ID NO: 15) are shown. The mRNA-translation start sites (ATG) are marked by black arrows. The corresponding amino acid sequence is denoted in SEQ ID NO: 16. For each gene, the upper sequence is in the sense orientation and the lower, antisense, complementary sequence served as the target DNA strand, which was barcoded and subsequently amplified by the MEMDS protocol. Positions that vary between the two genes are marked by circles below the HBD segment. Positions marked by filled circles were used to sort NGS reads from the same sperm-DNA sample to separate HBB and HBD datasets at the sequence analysis stage, as the two genes were barcoded and amplified simultaneously by the MEMDS procedure. The Bsu36I (RE-1)-recognition sequence is marked by a frame and its cleavage sites are marked by small black triangles. Position 20, where the HbS (20 A to T) mutation occurs, is marked by a curved arrow. The base denoted by a lower-case letter in the center of the Bsu36I site can tolerate any substitution without affecting Bsu36I activity. Therefore, the region of interest (ROI) is confined to six of the seven bases in the frame that constitute the Bsu36I site. The HpyCH4III (RE-2)-recognition sequence is also marked by a frame and its cleavage sites are marked by small black triangles. The base denoted by a lower-case letter in the HpyCH4III site can tolerate any substitution without affecting HpyCH4III activity. The sequence in the left-hand box anneals to oligo A and receives the primary barcode via a single, fill-in reaction (see FIG. 1B). Note that the first base that primes this extension, marked by a lower-case letter, differs between HBB and HBD. Therefore, a mixture of oligo A sequences that carry either one of the two complementary bases was used to minimize any bias due to delayed extension by the Q5 DNA polymerase. The sequence in the right-hand box anneals to oligo C and receives the secondary barcode via a single extension reaction (see FIG. 1E). The sequence between oligo A and oligo C remains untouched by any primer and therefore is suitable for mutation detection analysis. Yet only mutations at the ROI can be enriched, while mutations in the flanking right (R) and left (L) sequences are unlikely to affect Bsu36I digestion.

FIG. 4 is a graph showing the percent Bsu36I-resistance for a synthetic dsDNA library of HBB gene segments containing the Bsu36I-restriction site with its flanking sequences, and a single point mutation per segment in which a single base was substituted. The six bases that constitute the HBB ROI are shown in boxes and the identities of the substituting bases (T, C, A, G) are color-coded.

FIG. 5A-FIG. 5B is a graph showing the frequency of erroneous barcode labeling. FIG. 5A: Frequency of indirect labeling by the primary-barcode oligo (oligo A) as measured by the fraction of reads carrying the control guanine insertion. FIG. 5B: Frequency of secondary-barcode primer (oligo C) relabeling as measured by the relative frequency of reads carrying the sequence signature of the control secondary barcode relabeling primer (oligo D).

FIG. 6 are graphs showing family-size distributions. Distributions of primary-barcode families based on the number of read in a family (family size). In red: counts of families with primary barcode sequences that deviate by a Hamming distance of one from primary barcode sequences of families with a greater number of reads. In green: counts of families with primary barcode sequences that deviate by a Hamming distance>1 from primary barcode sequences of families with a greater number of reads. Different scales are used for the Bsu36I-untreated and treated samples. (The differences in family size between the two treatments are merely due to the higher recovery of ROI families in the Bsu36I-untreated samples, which lack depletion of wildtype sequences.)

FIG. 7A-FIG. 7C are graphs showing effects of various cutoff criteria on mutation-calling accuracy. Upper row: average values from the Bsu36I-treated samples of AFR1, AFR2, EUR1, EUR2. Lower row: average values from the Bsu36I-untreated samples of the same donors. Bar graphs: error rate per base in log-10 scale (left axis) while varying each cutoff criterion alone, calculated for the 47 bp that constitute the HBB and HBD ROI-flanking sequences for the Bsu36I-treated samples and for the 54 bp that constitute the ROI and the flanking sequences for the Bsu36I-untreated samples. Dot plots: Fraction of HBB and HBD families that meet a cutoff criterion (right axis). FIG. 7A: The effect of increasing the family-size cutoff. Mutations present at 100% of the sequences in a primary-barcode family were selected for the mutation-rate calculation. FIG. 7B: The effect of increasing the mutation-frequency cutoff for families with at least four reads. FIG. 7C: The effect of increasing the secondary-barcode count cutoff for families with at least four reads.

FIG. 8 is an illustration of the MEMDS computational pipeline. Abbreviations: BC1—Primary barcode; BC2—Secondary barcode; i—Family index; Si—Number of reads in family i (family size); k—Position in sequence; j—Mutation at position k; T1—Family-size cutoff; T2—Mutation-frequency cutoff; T3—BC2-count cutoff; Pj,k—Fraction of reads within family i with mutation j at position k; B_{j,k}—Number of unique BC2 barcodes in family i associated with mutation j at position k; P_{wt,k}—Fraction of reads within family i with wildtype (wt) base at position k; B_{wt,k}—Number of unique BC2 barcodes in family i associated with the wt base at position k; N—Ambiguous base when neither the mutation nor the wt passed the T2 and/or T3 cutoff.

FIG. 9 are graphs showing percent recoveries of WT (genomic) ROI sequences and artificial (plasmid) ROI sequences in BsuI-untreated and treated HBB and HBD.

FIG. 10A-FIG. 10B are graphs showing calculated error rates. FIG. 10A: Per-base error rates for non-G to T, C to T and C to A mutations (in the target DNA strand) were calculated for each donor for the 47 bp that include the ROI-flanking sequences in the Bsu36I-untreated samples (black bars) and Bsu36I-treated (gray bars) samples, under the stringent assumption that all mutations observed in these unenriched sequences are errors. Open circles mark samples where no non-G to T, C to T and C to A mutations were observed and the error rate calculation for these samples used a theoretical mutation count value of 1. FIG. 10B: The total error rate for each gene (i.e., the sum of non-G to T, C to T and C to A mutations for that gene across all donors divided by the total number of bases) calculated for the same sequences depicted in (A).

FIG. 11 is a graph showing per-type point mutation frequencies. In gray: mutations in the target (antisense) DNA strand. In black: the complementary mutations in the sequenced (sense) strand.

FIG. 12 is a graph showing mutation distribution in HBB and HBD sequences. Shown are the total mutation frequencies in HBB (left) and HBD (right) sequences of all 11 donors. Frequencies of mutations from the Bsu36I-treated and untreated samples are displayed in opposite directions. The Bsu36I-restriction site, six of whose seven bases define the ROI, is boxed by dashed lines. Both HBB and HBD sequences are shown in the sense orientation, which corresponds to the sequencing output data. Since this MEMDS experiment targeted the antisense strand of both genes, the mutations in the target DNA molecules were the reciprocals of the mutations shown here.

FIG. 13 is a graph showing correlation between the enrichments of target-strand G to T and C to T mutations and the Bsu36I-enrichment factors. The fold enrichment of the ROI G to T (filled circles) and C to T (open circles) mutations (C to A and G to A mutations in the sequence data, respectively) was determined by the ratio between the mutation frequencies in the ROI of the Bsu36I-treated and untreated samples. For each mutation type data is shown only for donors with at least 3 mutation counts in the ROI site.

FIG. 14 is a schematic illustration of the experimental design combining WT depletion with ddPCR to compute mutation frequency. Abbreviations: [S]—Concentration of genomic DNA (mostly RE-1-sensitive) in source tube; [R]—Concentration of artificial DNA (RE-1-resistant) in source tube; V_{Se}—Volume taken from “S” source tube to the RE-1-treated sample; V_{Sc}—Volume taken from “S” source tube to the RE-1-untreated sample; V_{Re}—Volume taken from “R” source tube to the RE-1-treated sample; V_{Rc}—Volume taken from “R” source tube to the RE-1-untreated sample.

### DETAILED DESCRIPTION OF EMBODIMENTS

The present disclosure concerns a method of measuring the origination rates of target mutations of choice. The method enables the measurement of mutation rate variation at an exceedingly high resolution across loci reaching a sequencing accuracy of about a hundred-fold higher than other sequencing methods known in the art.

The present disclosure therefore provides an ultra-accurate, high-yield method of identifying genetic variants, including ultra-rare variants such as de novo mutations, in DNA from small to very large populations of cells, as well as cell-free genomic or synthetic DNA.

Removing as many wild-type sequences as possible prior to DNA sequencing, while measuring the extent of that removal, would greatly improve both accuracy and sequencing efforts.

Accordingly, one of the key features of the method of the present invention is the use of a digestion step to remove wild-type molecules. The digestion may be performed using any method known in the art for example using a restriction-enzyme or a CRISPR-CAS9 system. Another key feature of the method is the use of a control condition which allows the calculation of the number of molecules scanned and thus the denominator for the mutation rate.

The method thus provides high accuracy in handling large amounts of DNA, while substantially reducing sequencing costs and increasing yield and is therefore broadly applicable, specifically to the analysis of the human genome.

In general terms, the method of the invention combines a step of quantified mutation enrichment with a step of mutation detection.

Namely, after the step of removing the wild-type molecules (i.e., the mutation enrichment) while calculating the amount of wildtype molecules that were removed, a next step is performed in which the mutation is identified. This identification may be done using sequencing methods, for example, but not limited to barcoding-based sequencing or using an alternative non-sequencing-based identification method including, but not limited to qPCR (quantitative Polymerase Chain Reaction) or ddPCR (digital droplet Polymerase Chain Reaction).

### Mutation Enrichment Combined with Barcoding-Based Sequencing

Therefore, in one aspect, one method of the invention, also referred to herein as MEMDS (Mutation Enrichment followed by upscaled Maximum Depth Sequencing), reaches a notably higher accuracy than MDS at a much smaller cost while focusing on detecting mutations in a very narrow ROI. The method of the invention enriches the sample for mutations in the ROI prior to library preparation by removing a large fraction of non-mutated variants. In addition, it includes various steps that a) further enable the processing of the very large initial amounts of genomic DNA, as required for identifying de novo mutations in humans; b) enhance accuracy by using routinely and in an informed manner a dual barcoding system and other measures guarantying the authenticity of target DNA molecules; and c) accurately quantify the fraction of non-mutated variants removed, which is necessary in order to obtain the denominator for the calculation of de novo mutation rates. Using this method of the invention, an error rate of at least 2.5×10⁻⁹ per base was achieved after removing the high-frequency G to T, C to T and C to A mutations (see Example 8) and a recovery rate of ˜35% of the input target sequences due to normal loss of material. With this recovery rate, for example, starting with 3 instances of a specific mutation in 300 million cells, 1 mutation in 100 million cells on average could be identified and reported. Thus, the recovery rate only affects the cost of sampling, and does not affect the cost or the accuracy of sequencing.

### The Mutation Enrichment Combined with Barcoding-Based Sequencing Outline

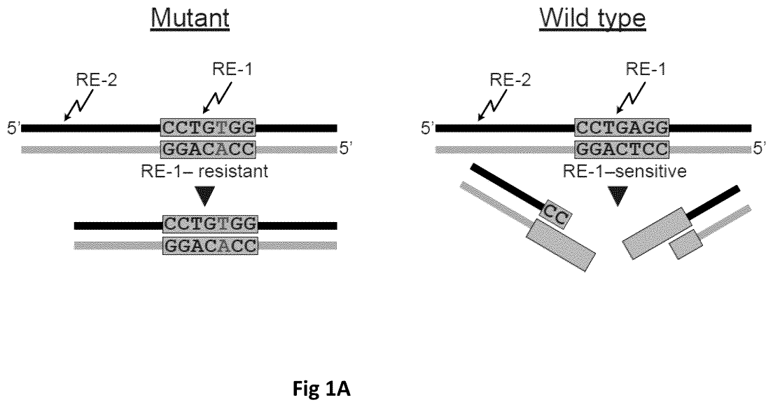

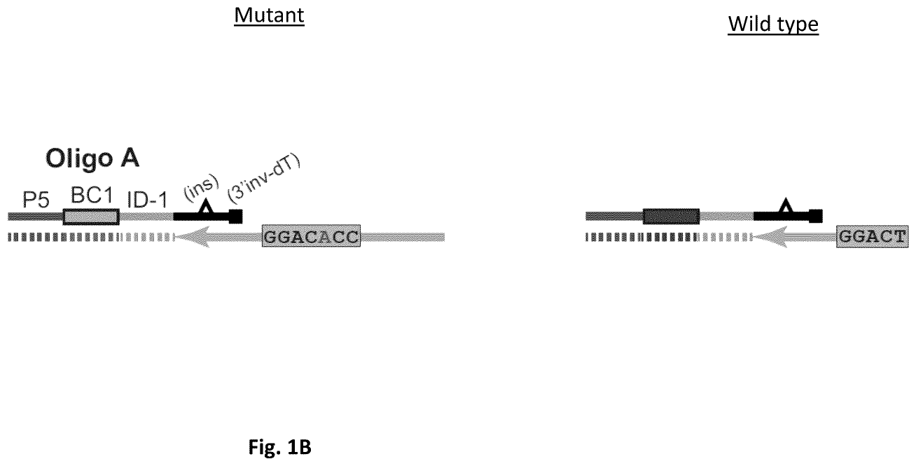

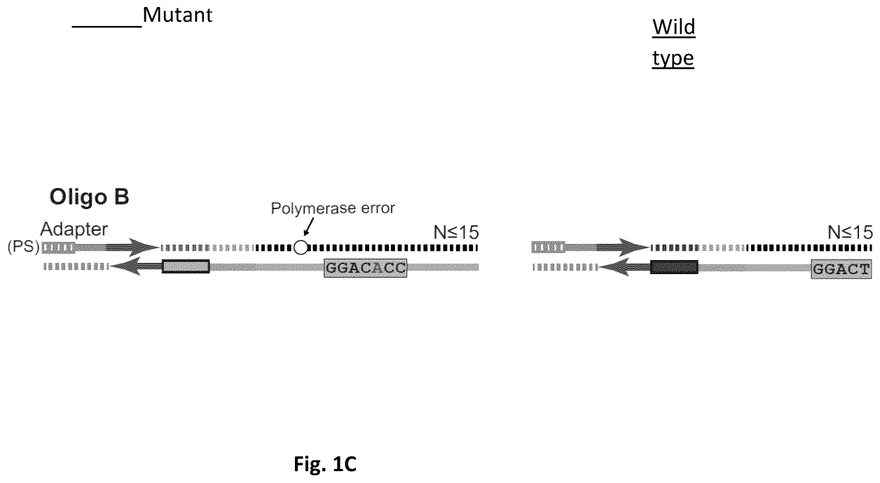

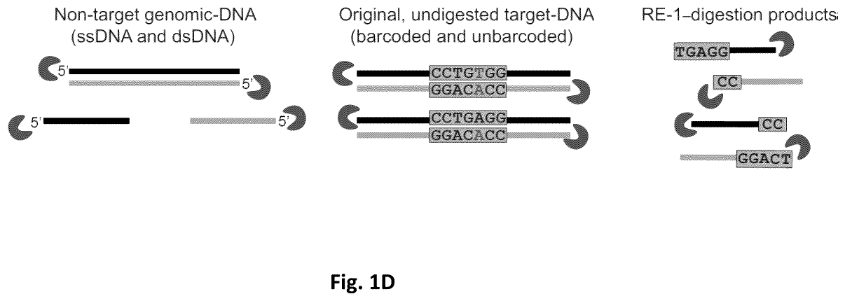

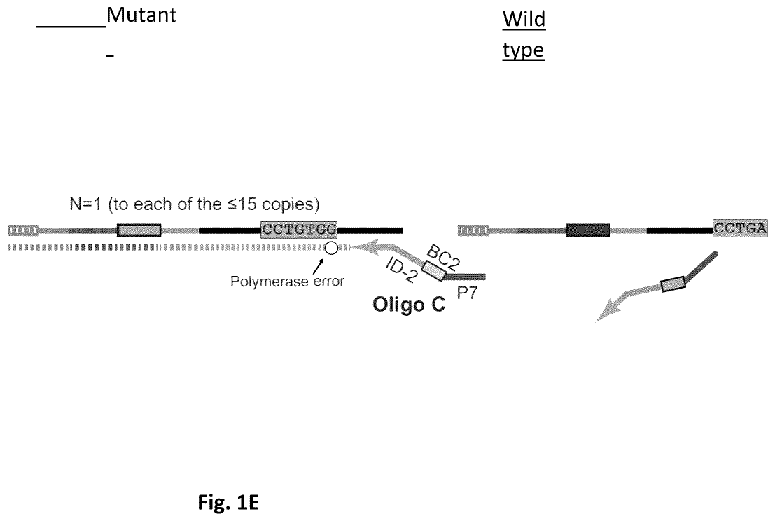

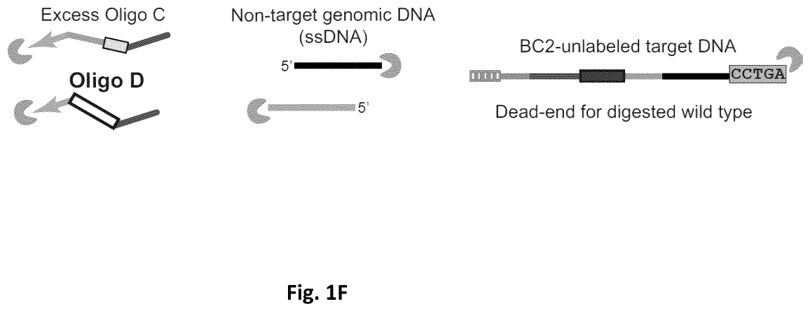

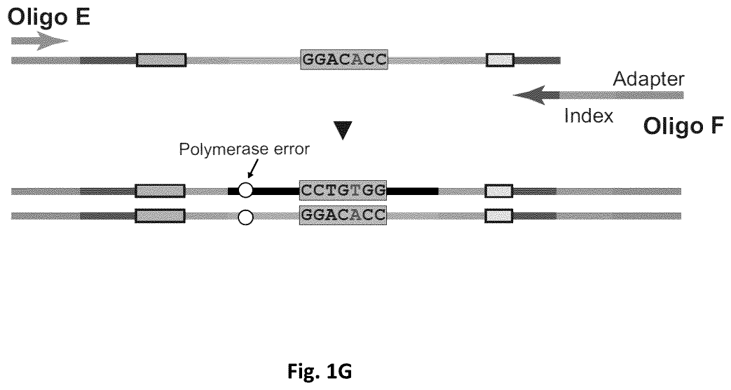

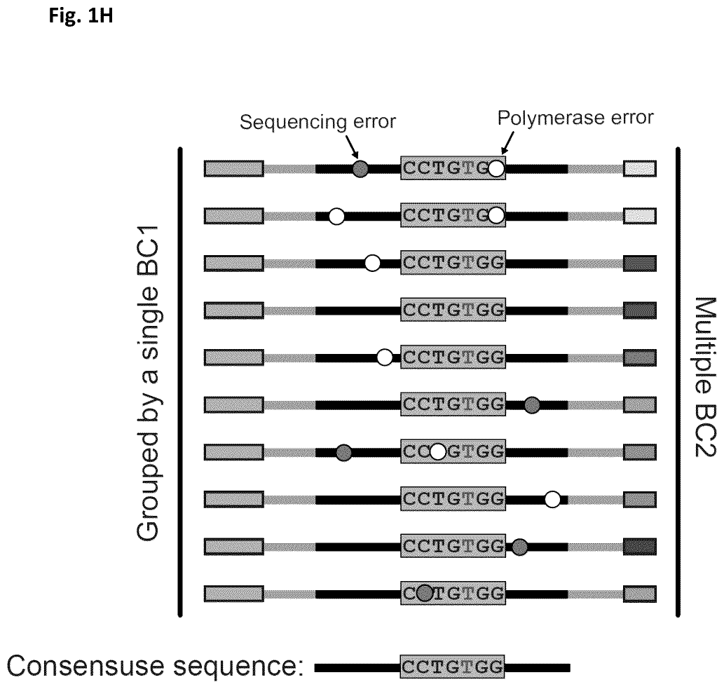

This aspect of the invention involves two workflows that are run in parallel. One enriches for mutations at the ROI using for example restriction enzyme digestion or CRISPR-editing (Jinek M, et al. (2012) Science 337(6096):816-821) depending on the types of mutations that are sought after (point mutations, indels) and the improvement in site-recognition specificity. The other workflow is used for computing the enrichment fold, and hence the exact number of wild-type ROI sequences that were removed from the ROI pool. The protocol outlined below and in FIG. 1 describes the workflow for the enrichment of mutated ROIs. This workflow is identical to the one applied for computing the enrichment fold, with the exception that the restriction enzyme used for enrichment (FIG. S1, step 1) is omitted in the latter. For a detailed explanation of the complete experimental design involving the two workflows, see Examples 1 and 2.

It should be emphasized that the methods of the invention are suitable for detection of any type of DNA (e.g., genomic DNA, cell-free DNA) obtained from any kind of sample. However, for illustrative purposes the Examples refer to genomic DNA.

The method is illustrated in FIG. 1, referring as a non-limiting example to genomic DNA.

Briefly, FIG. 1A: Step 1) Enzymatic digestion of genomic DNA. Restriction Enzyme-1 (RE-1) digests a region of interest (ROI) with a wild-type sequence and is blocked by a mutation at this site. Restriction Enzyme-2 (RE-2) digests closely to the ROI. FIG. 1B: Step 2) A fill-in reaction is promoted by oligo A, which anneals to the sequence between the RE-1 and RE-2 sites and introduces directly to the target DNA strand (gray strand) a sample-identifier sequence (ID-1) common to all labeled sequences in the sample, a primary-barcode sequence (BC1) that is unique to each target DNA molecule and an Illumina-P5 sequence. Shown also are the 3′ inverted-dT (3′inv-dT) that blocks oligo A extension and the control base-insertion (ins) for the identification and removal of extension products from unblocked oligo A at the sequence analysis step. FIG. 1C Step 3) Linear amplification of the barcoded target molecules is carried out for 15 cycles using oligo B that anneals to the P5 sequence of the target DNA strand and adds an Illumina adapter sequence with 5 phosphorothioate bonds at the 5′ edge (5′PS) of each barcoded-ROI copy. This linear amplification reaction results in 15 or less copies of each barcoded target molecule (N≤15). While polymerase errors (marked by circles) do occur, they are unlikely to repeat themselves at the same position in multiple copies of the same target molecule. FIG. 1D: Step 4) A mixture of 5′ exonucleases (“pacman” symbols) is added to degrade from 5′ to 3′ non-target genomic DNA including RE-1 and RE-2 digestion products. The barcoded copies are protected from this degradation step due to their 5′PS bonds. FIG. 1E: Step 5) A single-extension reaction with oligo C is carried out to add to each linearly amplified copy of a BC1-labeled target molecule an additional sample identifier sequence (ID-2), a unique secondary-barcode sequence (BC2), and an Illumina-P7 sequence. For each target molecule, this step results in 15 or less copies that share the same primary barcode at one end, while having a unique secondary barcode at the other end. Since oligo C anneals 3′ to the ROI sequence, any linearly amplified copy of RE-1 digestion product cannot be labeled by this oligo. FIG. 1F: Step 6) A 3′ exonuclease (“pacman” symbol) is added immediately after the single-extension reaction to degrade from 3′ to 5′ any single-stranded DNA, including excess of oligo C, to prevent secondary-barcode relabeling during the next PCR reaction. Copies labeled by secondary barcodes are protected from this degradation step due to their double-stranded state, while non-labeled copies are single-stranded and are therefore degraded. A relabeling-control primer (oligo D), carrying a unique sequence signature, is added in known amount together with the 3′ exonuclease to assess at the sequence analysis step, the number of oligo C relabeling in the event of incomplete degradation of oligo C by the 3′ exonuclease. FIG. 1G: Step 7) PCR amplification completes the final sequence requirements for Illumina NGS and produces a library of barcoded ROI sequences composed of enriched mutation variants as well as wild-type sequences that escaped RE-1 digestion. FIG. 1H: Step 8) Following next-generation sequencing, reads are grouped into families based on their primary-barcode sequences, so that within each family, all members have the same primary barcode, and the consensus sequence for the family is determined using three parameters: family size, mutation frequency, and the number of secondary barcodes associated with each base. This procedure allows to eliminate PCR errors (empty circles) and NGS errors (filled circles), which usually appear in low frequencies and are linked to single secondary barcodes, and to accept as true, de novo mutations only mutations that appear in multiple reads and are associated with multiple secondary barcodes, such as the “T” substitution in the figure.

Step 1: Enzymatic digestion of genomic DNA: The genomic DNA is digested by two restriction enzymes. The first (RE-1) digests the wild-type sequence at a certain site that is several residues long and that constitutes the region of interest (ROI). Namely, the experiment is designed by choosing an ROI and a RE-1 so that the recognition site for RE-1 matches the wild-type sequence at the ROI. As a result, sequences with no mutations are efficiently digested, while variants with mutations that hamper site-recognition by RE-1 are protected from cleavage. These mutations are therefore enriched in the pool of un-cleaved sequences. The second restriction enzyme (RE-2) is used to cleave the DNA near the ROI. The choice of a suitable RE-2 is dependent on the availability of an adequate recognition site far enough from the RE-1 site to allow for an efficient annealing of a primary-barcode oligo (oligo A) between the two sites, yet short enough to meet the read-length limits of the chosen NGS sequencing platform. To satisfy these conditions, the RE-2 site may be selected to be either upstream or downstream of the ROI, a choice which will determine which of the two DNA strands will be barcoded and analyzed.

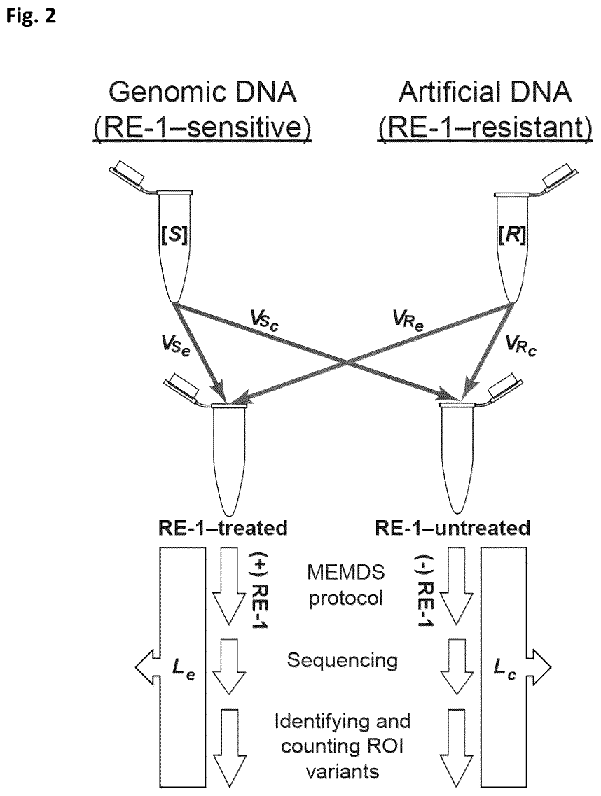

The exact number of wild-type ROIs that have been removed by RE-1 is calculated as shown in FIG. 2 and as detailed in Example 1.

Briefly, two tubes, one containing genomic DNA that carries mostly RE-1-sensitive ROI sequences, denoted S, and one containing artificial-ROI sequences resistant to RE-1 digestion, denoted R, are used as source tubes from which volumes are drawn in known amounts to create two mixtures of the two samples, designated “RE-1-treated” and “RE-1-untreated” samples (see the figure legend for the abbreviations used). These two samples undergo the full protocol, with the exception that the former is treated with and the latter without RE-1. Following next-generation sequencing, variants are identified by the method' computational pipeline and the numbers of RE-1-sensitive ROI variants (i.e., wild-type ROIs) and artificial RE-1-resistant ROI variants are determined for each sample (S^{f}ₑ and R^{f}ₑ denote the numbers of sensitive and resistant variants identified for the RE-1 treated sample, and S^{f}_{c} and R^{f}_{c} denote the sensitive and resistant variants identified for the RE-1 untreated sample). These numbers, together with the known volumes taken from the source tubes to create the input mixtures, are used to calculate the RE-1-enrichment factor, E, and the total number, W, of wild-type sequences that were either digested by RE-1 or evaded digestion and were sequenced and identified.

Step 2: Primary barcode attachment: Following digestion, the DNA is subjected to single-strand extension using a high-fidelity DNA polymerase and a single oligonucleotide (oligo A). Oligo A anneals with its 3′ part to the sequence between the RE-2 site and the RE-1 site and acts as a template for extension of the target-DNA strand. This extension reaction introduces three sequence features directly into the target strand: a) a segment of four bases that serves as a sample-identifier sequence to secure the sample in the event of a rare contamination by DNA libraries from other samples; b) 14 randomized bases that create a primary barcode unique to each specific DNA fragment; and c) an Illumina P5-primer sequence. To prevent the oligonucleotide itself from being extended while using an already barcoded target strand as template in the subsequent linear amplification step, an inverted-dT modification is included at the 3′ terminus of oligo A that blocks the DNA polymerase and prevents the extension of oligo A during the process. To account for the event that some oligo A molecules escaped the inverted-dT modification during their synthesis by the manufacturer, a single-base insertion is planted in the oligo A sequence that anneals to the genomic strand, so that undesired extensions of rare, unblocked oligos could be easily detected at the sequence analysis step for their inclusion of this single-base insertion and removed.

Step 3: Linear amplification of barcoded ROI products: The genomic ROI is linearly amplified by 15 cycles using a high-fidelity DNA polymerase and a single primer (oligo B) that anneals to the Illumina P5-primer sequence. Oligo B contains the complete Illumina-adapter sequence and carries five phosphorothioate bonds (PS) at its 5′ edge. This step results in up to 15 single-stranded copies of each barcoded ROI (each copy having been generated directly from the same barcoded original DNA molecule), protected by the phosphorothioate bonds from 5′-exonuclease activity.

Step 4: Degradation by 5′-exonucleases: The linear amplification products are treated with a mixture of 5′-exonucleases, which degrade both single and double-stranded DNA with or without phosphate groups at their 5′ termini, from the 5′ edge to the 3′ edge of each strand. The linearly amplified ROI copies are protected from this exonuclease activity due to the multiple PS bonds at their 5′ edges. This step removes most of the genomic DNA, including most of the ROI digestion products, and simplifies the rest of the experimental workflow by allowing the next reactions to be carried in a small number of tubes rather than in 96-wells plates as well as by eliminating sequences that could potentially promote the generation of unwanted byproducts in the subsequent amplification steps.

Step 5: Secondary barcode attachment: The DNA from the 5′-exonuclease reaction is subjected to a single primer-extension reaction, using a secondary-barcode primer (oligo C) that anneals 3′ to the ROI site and extends by a single cycle using a high-fidelity polymerase. The secondary-barcode primer also carries three features: a) a segment of four bases that serves as a sample-identifier sequence; b) five randomized bases that create a secondary barcode generally unique to each member within a group of copies (copies sharing the same original DNA molecule); and c) an Illumina P7-primer sequence. This step produces a complementary strand for each of the 15 copies (or less) generated per target-DNA molecule during the linear amplification step. Each of these complementary strands carries the same primary-barcode sequence and a unique secondary-barcode sequence.

Step 6: Degradation by a 3′-exonuclease: To prevent recurrent labeling by secondary barcode primers in subsequent amplification reactions, a 3′-exonuclease that degrades single stranded DNA from the 3′ edge to the 5′ edge of the molecule is added immediately after the secondary barcode attachment to eliminate free, unbound primers. The double-stranded molecules that just completed the secondary barcode extension reaction are protected from this degradation. The 3′-exonuclease is added together with a known amount of relabeling control primer (oligo D). This control primer is identical in sequence to the secondary barcode primers except for the sample-identifier and the secondary-barcode features that are replaced by a known sequence. Therefore, in the event of incomplete degradation by the 3′-exonuclease, the amount of NGS reads with an oligo D sequence signature serves as a proxy for the frequency of relabeling by the secondary-barcode primer.

Step 7: Amplicon generation by PCR for next generation sequencing: PCR amplification of the purified DNA is carried using primers E and F, which add Illumina index and adapter sequences to the 3′ edge of the amplicon. Optionally, this step may be broken into two PCR reactions to preserve some of the first PCR product as a backup. [see for example the Materials and Methods section below] Importantly, RE-1 digestion products that were not eliminated until this step will not be amplified, as only complete segments that were not digested by RE-1 have the two primer annealing sites.

Step 8: Analysis of sequenced data: NGS reads are grouped into families based on their primary-barcode sequences. Thus, each family is made of a collection of sequences originated from linearly amplified copies of a single target-DNA strand, belonging to a single gene. Each read in a family is aligned against a reference sequence specific to the donor and mutations with a high-quality sequencing score are noted. Three criteria are then used in combination to select for true mutations: a) the number of reads in the family (i.e., family size); b) the number of secondary barcodes associated with a particular mutation (i.e., BC2 count); and c), the fraction of the specific mutation in the family (i.e., mutation frequency). Mutation candidates that pass the combined cutoff criteria are designated true, de novo mutations. The total number of target wild-type sequences screened, which consist of a) target wild-type sequences that were digested by RE-1 and removed from the final DNA libraries, and b) target wild-type sequences that evaded RE-1 digestion and were included in the sequenced DNA libraries, is calculated from the sequencing outputs of the RE-1-treated and the RE-1-untreated samples (see Examples 1 and 2 and FIG. 2 for a detailed description of this methodology). Finally, from the mutation count and the total number of cells scanned, the per-locus, per-mutation de novo mutation rate is calculated for mutations of interest in the ROI.

As shown in the Examples below, the method is described with respect to the HbS and nearby mutations in hemoglobin β (HBB) as well as to the equivalent mutations in the nearly identical delta-globin (HBD) gene in sperm cells from African and European donors. However, the method may be generally applied to any selected target gene. The inventors show that the HbS mutation originates de novo ˜35 times faster than expected from the genome-wide average (GWA) mutation rate for its type, specifically in the African donors. No HbS mutation was observed in a similar number of cells from the European donors and no HbS-equivalent mutation was observed in HBD in any donor. Out of 12 types of point-mutations observable in the African HBB gene region studied, the HbS mutation deviates most from its GWA. More generally, mutation-specific origination rates, unexplained by the local genetic context, vary between individuals and between populations in a manner that corresponds to observations of alleles in populations.

Different mutations in HBB that protect against malaria are known to have occurred and to have spread in human populations multiple times. HbS, the most notable mutation variant associated with malarial resistance, is a single base substitution (20 A to T) in codon 6 of the HBB coding sequence that causes a Glutamate to Valine change. Some other point mutations and short deletions near the HbS site are also known to confer malarial resistance. Delta-globin, encoded by the HBD gene, is expressed in adulthood together with HBB. These two paralogues exhibit a high degree of homology, showing 80% identity in coding sequence and 93% identity in amino acid sequence. However, mutations in HBD are not considered to be protective against malaria, probably due to its low expression levels compared to HBB, which accounts for less than 3% of the hemoglobin in adults.

### Mutation Enrichment Combined with ddPCR

In another aspect of the invention, the genetic variants are identified using ddPCR.

Droplet digital PCR (ddPCR) is a method that enables quantification of rare target DNA variants by partitioning the DNA sample into thousands of nanoliter-sized droplets and performing separate PCR amplification reaction in each droplet. Following PCR amplification, each droplet is analyzed individually using a two-color detection system that allows the identification of a specific mutant variant that is present at a very low frequency in a large pool of wild-type (WT) variants. The PCR amplification reaction involves two competitive probes, one for detecting the WT variant and one for detecting the mutant variant, each producing different color reading upon amplification. ddPCR can currently detect mutant DNA present at 0.1% in a background of WT DNA using ˜15,000-20,000 droplets per well. Increasing sensitivity beyond this level requires re-running multiple samples.

ddPCR is commonly used today, for example for medical diagnosis to detect and quantify cancer signature mutations in biopsies.

In accordance with this aspect of the invention, the DNA sample is digested using a restriction enzyme that recognizes a specific restriction site that matches the WT sequence. Such enzymes cut ˜98%-99% or more of the WT DNA while leaving DNA variants with mutations at the Enzyme's recognition site intact.

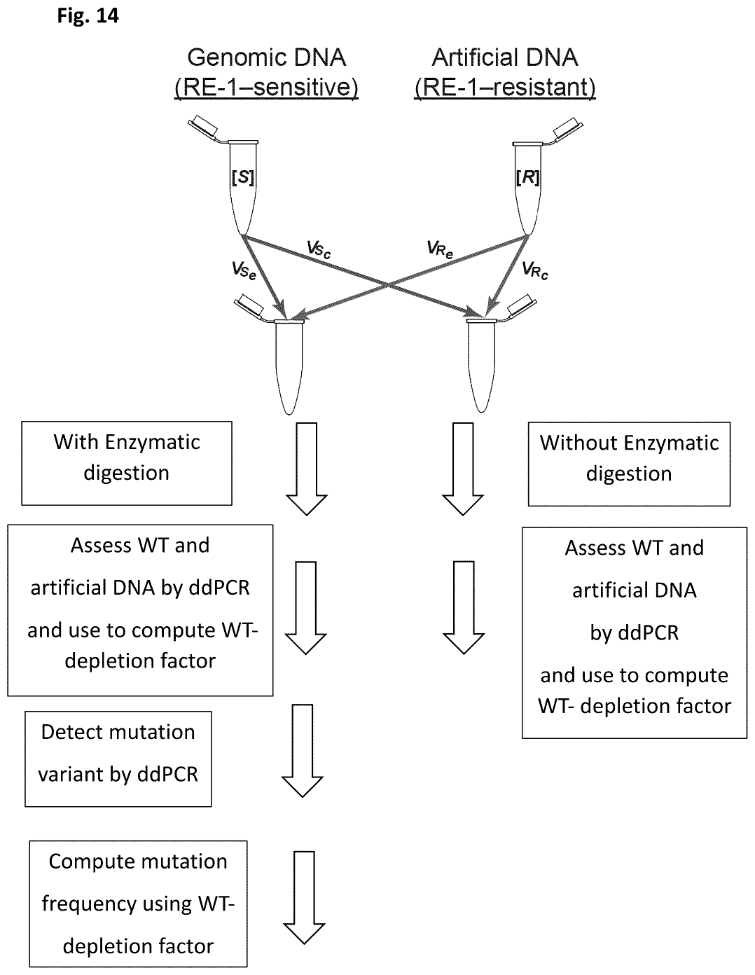

The method is illustrated in FIG. 14. The method is performed using two parallel reactions (referred to herein as restriction enzyme-treated and restriction enzyme-untreated reactions). Each reaction is supplemented with artificial DNA molecules in known amounts that are resistant to the enzymatic digestion. The two reactions undergo the same treatment except that the restriction enzyme is added only to the restriction enzyme-treated sample.

Given that, at the present time, only two probes can be used simultaneously in a ddPCR reaction, three ddPCR reactions are run: The first two reactions are carried on small aliquots from the restriction enzyme-treated and untreated samples with two specific ddPCR probes in each that can identify and count the occurrence of the WT and the artificial DNA molecules. By calculating the difference in artificial/WT DNA ratio between the two samples, the WT depletion factor by the restriction enzyme can be determined and the total number of WT sequences that were scanned by the enzyme can be calculated.

The third ddPCR reaction is performed on the remaining part of the restriction enzyme-treated sample to identify and count the occurrences of the mutation of interest. Two specific ddPCR probes are used to identify and count the WT and the mutant variant.

Optionally, if a larger number of probes can be used simultaneously, the number of ddPCR reactions could be reduced from three to two.

Without wishing to be bound by theory, use of the combination of mutation enrichment and ddPCR has the following benefit: starting with the same initial amount of DNA as would have been used for a regular ddPCR reaction, mutation enrichment reduces the number of wild-type molecules by ˜100 fold (with the precise fold depending on the specific target mutation/s and therefore the restriction enzyme used), thus increasing the accuracy of the procedure by ˜100 fold (due to reducing the probability of false positives emerging from incorrect reads of wild-type molecules).

The invention therefore provides in one of its aspects a method of identifying genetic variants in DNA; said method comprising:
a. Providing a sample of isolated DNA, wherein said DNA comprises wild-type DNA sequences and optionally one or more DNA sequences containing a genetic variant in one or more regions of interest (ROIs);
b. Removing said wild-type DNA sequences from said sample, thereby enriching the sample with DNA sequences containing the genetic variant;
c. Determining the number of the wild-type sequences that were removed by calculating an enrichment factor (E); and
d. Determining the number of genetic variants in said DNA.

As used herein the term “identifying” refers to discovering or evidencing the presence of a genetic variant in a DNA sequence present in a biological sample.

As used herein the term “genetic variants” refers to any changes in the DNA strands, e.g., a substitution of a nucleotide typical of the “wild-type” version of a DNA strand with another non-typical nucleotide (may also be referred to as a point mutation), as well as an insertion or a deletion of one or more nucleotides. A genetic variation is also referred to herein as a mutation.

The method of the invention may be practiced with any type of DNA (deoxyribonucleic acid), encompassing but not limited to, isolated genomic DNA as well as synthetic DNA (e.g., artificial DNA sequences).

As used herein the term “genomic DNA” refers the DNA originating from the genome of an organism or a virus and may encompass a part of said genome or a whole genome.

As used herein the term “organism” refers to any living entity such as a bacterium, fungus, plant, or animal. The term further encompasses any type of animal. In one embodiment, the term refers to a human.

The genomic DNA may be isolated, extracted or purified from a cell lysate originating from a biological sample. Several techniques and commercially available products are known in the art for performing DNA isolation, e.g., as described in Materials and methods. Isolated genomic DNA may also be purified from a bodily fluid, i.e., by purification of cell free DNA from the bodily fluid. The DNA may also be obtained from a non-biological source, for example but not limited to soil, sewer, water reservoirs, and any kind of surface.

Accordingly, a sample suitable for use in the method of the invention is a biological sample comprising cells of the tested organism or comprising viruses. The sample may be obtained from any bodily fluid e.g., sperm (semen), amniotic fluid, blood, cerebrospinal fluid, ascitic fluid, saliva, urine, bronchoalveolar lavage fluid, or nasal lavage fluid, or from a tissue biopsy. The biopsy may be taken from any tissue in the body, including a malignant tissue such as cancer. The sample may comprise a cell population of between about ≤10,000 cells and about 1×10⁹ cells. The sample may be fresh or frozen.

As noted above, a sample suitable for use in the method of the invention is also a non-biological sample, namely a sample obtained from a non-biological source, for example but not limited to a soil sample, sewer sample water sample, and a sample taken from any kind of surface.

As used herein the term “Region of interest” (ROI) refers to a stretch of DNA which may harbor a mutation and may serve as a basis for differentiating between a wildtype version of the DNA and a mutated version. This stretch may form a recognition site for a restriction enzyme (e.g., the Bsu36I site).

As used herein the term “removing” wild-type DNA sequences from the sample, refers to a process of eliminating the wild-type sequences from the sample using any means that allows for specific removal of the wild-type sequences while the corresponding sequences that harbor a mutation, a genetic variant, are maintained. Such elimination results in enrichment of the sample with DNA sequences containing the genetic variant. The removal of the wild-type sequences can be performed by any suitable method, for example by subjecting the DNA to a cutting agent that acts differently when encountering a wild-type sequence or a genetic variant.

As used herein, the term “enrichment factor” (designated E) refers to a calculated value which reflects the number of the target wild-type sequences that were removed and therefore the degree of the genetic-variants enrichment in the tested DNA. The enrichment factor may be calculated as described herein.

The term “subjecting” used herein refers to bringing together and maintaining a biochemical system e.g., DNA and a cutting agent (e.g., a restriction enzyme) under specific conditions suitable to promote a particular reaction.

As used herein the term “cutting agent” refers to any molecule or combination of molecules that can cleave DNA at a predefined location. The cutting agent may be an enzyme, e.g., an endonuclease or a restriction enzyme. A restriction enzyme is an enzyme that cleaves DNA into fragments at specific recognition sites, i.e., specific nucleotide sequences. A very large number of commercially available restriction enzymes are known in the art for performing cleavage of DNA at specific locations, e.g., as described in Materials and methods.

The cutting agent may also be a CRISPR-Cas9 agent.

As used herein the term “barcode” refers to a DNA barcode, which is a unique, identifiable DNA sequence tag that is attached to a target DNA fragment and is used to identify the target DNA fragment during DNA sequencing.

In the context of the present invention, direct attachment of a primary barcode is done using an oligonucleotide (e.g., oligo A as outlined in the Examples below). Optimal length of each of the components of the oligonucleotide varies depending on the platform used. None of the length or the composition of the barcode sequence are limiting on the invention.

The term “primer” as used herein refers to an oligonucleotide which acts as a point of initiation of nucleic acid synthesis when placed under suitable conditions. Synthesis of a primer extension product, a nucleic acid which is complementary to a template strand, is induced in the presence of nucleotides and an agent for polymerization, such as a DNA polymerase enzyme, at a suitable temperature and pH. Primers used in this invention may be comprised of naturally occurring dNTP, modified nucleotides, or non-natural nucleotides. Primers must be sufficiently long to prime the synthesis of extension products in the presence of the agent for polymerization. The exact length of the primers will depend on many factors, including temperature, application, and source of primer. Neither the length, nor the composition of the primer are limiting on the invention.

In one embodiment, a primer forms part of an oligonucleotide designed to attach a barcode (e.g., oligo A).

In other embodiments, the primers of the invention are used for performing a polymerase chain reaction (PCR) with the amplified barcoded ROI products.

As used herein the term “Next Generation Sequencing (NGS)” refers to a DNA sequencing technology, based on a unique NGS machinery (e.g., the Illumina NGS platform).

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

### EXAMPLES

### Example 1

### Calculating the Number of RE-1 Digested Sequences

The evolutionarily relevant de novo mutation rate of a mutation in a sample is the number of target sequences in the sample identified as carrying that mutation divided by the total number of target sequences scanned by this method of the invention. The number of target sequences scanned by this method of the invention includes two sets of molecules: a) target sequences identified at the sequence analysis step; and b) target sequences removed by the enrichment step as described above (FIG. 1A). Therefore, to calculate the mutation rate, one must be able to determine how many target-wild-type sequences have been removed by RE-1 digestion as opposed to having been removed by general loss of genetic material during the procedure. The fold-reduction in target wild-type sequences, also referred to here as the RE-1 enrichment fold for RE-1-resistant mutations, multiplied by the number of target wild-type sequences identified at the sequence analysis step, yields the number of target sequences scanned by this method of the invention. This number, in turn, serves as the denominator in the mutation rate calculation.

Toward this end, an experimental design has been established that uses the NGS output to obtain a precise measurement of the RE-1 enrichment fold. This experimental design avoids errors due to impreciseness of input DNA concentration measurements and variation in DNA loss and in performance of the method steps across samples.

Two tubes are employed: a genomic- and a mock-DNA tube (FIG. 2). The genomic-DNA tube includes the DNA extracted from the human sperm sample (see Materials and Methods). In people who are not carriers of HbS or other mutations in the ROI, this tube contains mostly wild-type target sequences, which are sensitive to digestion by RE-1, and are denoted S. The other tube is a mock-DNA tube containing copies of an artificial sequence, denoted R, that are resistant to RE-1 digestion and easily distinguishable from natural mutants at the sequence analysis step. From the genomic-DNA tube, an amount of material is transferred into an “RE-1-treated” tube (FIG. 2), whose material will undergo the full protocol including RE-1 digestion, and another amount into an “RE-1-untreated” tube, whose material will undergo the same steps except for digestion by RE-1. Likewise, from the mock-DNA tube, an amount of material is transferred into the RE-1-treated tube and another amount into the RE-1-untreated tube. The principle underlying this design is that the relative amounts transferred from a tube can be known through their volume measurements alone and, given these measurements, the RE-1 enrichment fold can be obtained by comparing the ratios between the numbers of sensitive and resistant DNA molecules following each treatment (as shown below), where those amounts are precisely known from the sequence analysis step.

Specifically, let the concentrations of S in the genomic-DNA tube and of R in the mock-DNA tube be [S] and [R], respectively. From the genomic-DNA tube a volume V_{Se} is moved to the “RE-1-treated” tube and a volume V_{Sc} to the “RE-1-untreated” tube. From the mock-DNA tube a volume V_{Re} is moved to the RE-1-treated tube and a volume V_{Rc} to the RE-1-untreated tube. Lₑ represents the fold loss of material (whether sensitive or resistant) due to normal loss in the experimental condition, and L_{c} represents the fold loss of material due to normal loss in the control condition. E represents the RE-1 enrichment factor (i.e., 1/E is the fold reduction in sensitive molecules in the RE-1-treated tube due to RE-1 digestion). At the final, sequence analysis step, all the following can be counted: the number of sensitive (i.e., wildtype) molecules called in the RE-1-treated condition, S^{f}ₑ; the number of artificial, resistant molecules called in the RE-1-treated condition, R^{f}ₑ; the number of sensitive (i.e., wild-type) molecules called in the RE-1-untreated condition, S^{f}_{c}; and the number of resistant molecules called in the RE-1-untreated condition, R^{f}_{c}. These quantities can be presented by the following formulas:

$${S}_{e}^{f} = [ S ] \times {V}_{Se} \times {L}_{e} \times 1 / E ,$$

$${R}_{e}^{f} = [ R ] \times {V}_{Re} \times {L}_{e} ,$$

$${S}_{c}^{f} = [ S ] \times {V}_{Sc} \times {L}_{c} , and$$

$${R}_{c}^{f} = [ R ] \times {V}_{Rc} \times {L}_{c} .$$

Therefore, E can be obtained by using the following formula:

$$E = \frac{Rfe \times Sfc \times VSe \times VRc}{Sfe \times Rfc \times VRe \times VSc} \qquad ( 1 )$$

where all terms on the right-hand side are precisely known.

Given E, the number of wild-type molecules scanned by the procedure, W, namely molecules either removed by RE-1 (and are therefore wild-type) or identified as wild-type at the sequence analysis step, is

$$W = {S}_{e}^{f} \times E \qquad ( 2 )$$

Following is an example for the calculation of E and W based on DNA concentrations in each source tube and volumes taken for the input mixtures that are similar to the DNA concentrations and volumes used in the experiments but are rounded for the sake of simplicity here:

[S]=330 ng/μl of genomic DNA (˜100,000 genomes/μl)
[R]=10 fg/μl of artificial DNA (˜3,000 plasmids/μl)
V_{Se}=800 μl (˜80,000,000 genomes)
V_{Sc}=40 μl (˜4,000,000 genomes)
V_{Re}=20 μl (˜60,000 genomes)
V_{Rc}=120 μl (˜360,000 genomes)
S^{f}ₑ=200,000 wild-type variants
R^{f}ₑ=20,000 plasmid variants
S^{f}_{c}=400,000 wildtype variants
R^{f}_{c}=400,000 plasmid variants
Calculated values:

### E=120

### W=24,000,000

Lₑ=56,000,000 (70% loss)
L_{c}=3,600,000 (90% loss)

In this example, the RE-1-enrichment factor equals 120, meaning that de novo mutations in the ROI, which block RE-1 similarly to the artificial sequences, are enriched 120-fold in the RE-1-treated sample compared to the RE-1-untreated sample. Using this enrichment factor, the total number of unique wild-type molecules screened by the this procedure is 24,000,000, which includes the number of wild-type target molecules in the RE-1-treated sample that were digested by RE-1 and the 400,000 RE-1-sensitive variants that escaped digestion and were sequenced. Note that the calculation of E and W relies only on the number of original target molecules that were sequenced in the computational analysis step and on the volumes used to generate the input mixtures, and therefore the number of genomes and artificial sequences in the source tubes is not needed for it. Yet, by having a rough estimate of the actual amount of DNA transferred from the source tube, one can assess the number of target DNA molecules (either genomic or artificial ROI-including molecules) that were lost during the procedure of this method of the invention (not due to RE-1 digestion but due to general loss of material involving the efficiencies of labeling, amplifying, purifying, capturing, and sequencing all target sequences).

Thus, under the following assumptions, there is no need to know [S], [R] or the amount of material lost during the runs of the two parallel protocols in order to know the RE-1 enrichment fold: a) the solutions can be kept sufficiently homogeneous for the purposes of drawing volumes of similar concentrations from them (this is ensured by a thorough mixing before drawing); b) volumes, at the range used, can be measured easily and accurately; c) the normal loss of material during the run of the protocol (i.e., loss that is not due to RE-1 digestion) within any one treatment of a sample (RE-1-treated or untreated) does not substantially differ between sensitive and resistant molecules (as is confirmed by observation; see Example 7).

Finally, suppose that mutations of n different types have been identified in the ROI at the sequence analysis step (mutations that confer resistance to digestion by RE-1). Mᵢ represents number of instances of mutation of type ∈{1, 2, . . . , n} identified at that step. The rate of mutation i is then

$$\frac{{M}_{i}}{W + {\sum }_{j = 1}^{n} Mj} \qquad ( 3 )$$

Since the sum in the denominator is negligible compared to W, it suffices to calculate the rate of mutation i as

$$\frac{Mi}{W} \qquad ( 4 )$$

### Example 2

### Practical Considerations of Amounts Used for the Treated and Untreated Samples

While small amounts of mock-sequences could be easily identified in the RE-1-treated sample due to their enrichment, using the same amounts in the RE-1-untreated sample where no enrichment for RE-1- resistant variants is carried out would require a large sequencing effort to trace them among the vast numbers of wild-type sequences. On the other hand, using large amounts of mock-DNA would improve their sequence recovery in the RE-1-untreated sample but would consume NGS capacity at the expense of sperm sample sequences in the RE-1-treated sample. Similarly, while large amounts of genomic DNA can be used in the RE-1-treated sample due to the removal of many wild-type ROIs by RE-1 from the final sequencing input, using the same amount of genomic DNA in the RE-1-untreated sample will require a massive sequencing effort.

Therefore, to match the experimental design to the NGS coverage limitations, the following procedure is performed: From a single human-sperm DNA source a volume equivalent to ˜60-80 million haploid cells is transferred to the RE-1-treated tube, and a volume equivalent to exactly 5% of the initial amount taken for the RE-1-treated tube (i.e., ˜3-4 million haploid cells, respectively) is transferred to the RE-1-untreated tube. For each ROI to be analyzed, a mix of two linearized plasmids is used as the mock-DNA sample. These plasmids carry all the ROI-flanking sequences that are necessary for processing by the MEMDS protocol, and each is designed to carry a unique stretch of mutations at the ROI that distinguishes it from the wild type, from natural mutants, and from the other plasmid (multiple mutations are used to make it practically impossible for the plasmid to be indistinguishable from natural mutants). Using the same plasmid-mix source tube, a volume equivalent to 7,500 copies from each linearized plasmid is added to the RE-1-treated tube (thus creating a genome:plasmid ratio close to 10,000:1) and 45,000 copies from each plasmid to the RE-1-untreated tube (creating a genome:plasmid ratio close to 100:1). Importantly, and as discussed above, the relative volumes drawn from any one source tube, not the absolute amounts of genomic and plasmid DNA, are the values that matter for the RE-1 enrichment fold calculation (Eq. 1).

### Example 3

### HBB and HBD Sequence Features Utilized by the MEMDS Method

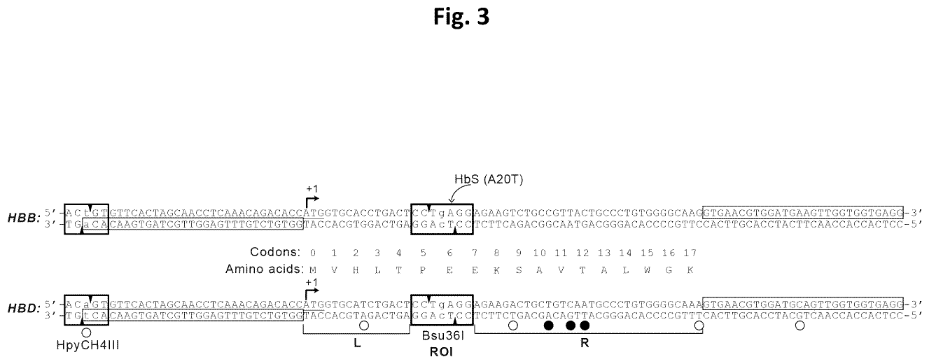

The HBB and HBD gene sequences that were selected for processing by the MEMDS method encompass 114 bases from exon 1, ranging from 32 nucleotides upstream of the mRNA translation start site to 81 nucleotides into the protein coding sequence (FIG. 3). This region is highly conserved between the two genes, which differ in only eight of the 114 bases. The region of interest (ROI) is a palindromic sequence found between positions 16-22 of the coding sequence, which forms the recognition site for the restriction enzyme Bsu36I (CCTNAGG) both in HBB and HBD. Since Bsu36I can tolerate any of the four possible nucleotides at the central position of its recognition sequence, the ROI is limited to six of the seven nucleotides of this palindromic sequence. Therefore, Bsu36I serves as RE-1, which digests non-mutated (wildtype) ROI sequences and enriches for HBB- and HBD-ROI mutation variants (FIG. 1A). The second restriction enzyme, HpyCH4III, which serves as RE-2 for the primary-barcode attachment, digests the HBB and HBD gene segments at its recognition site (ACNGT), 45 bases upstream of the 5′ edge of the Bsu36I restriction site. The identity of the “N” base at the center of the HpyCH4III site is of central importance, as after digestion by HpyCH4III this base is found at the 3′ terminus of the antisense strand that extends to incorporate the primary barcode via a fill-in reaction (FIG. 1B). Since HBB and HBD carry a different nucleotide at this “N” position of the HpyCH4III recognition site, the primary-barcode oligo (oligo A) that initiates the fill-in reaction carried a randomized base at that position, matching either one of the two complementary bases to allow for similar efficiencies of primary-barcode synthesis for the two genes.

A region of 30 bases between the Bsu36I and the HpyCH4III sites is used as the annealing site for the primary-barcode oligo, and a region of 28 bases starting 60 bases downstream to the 3′ edge of the Bsu36I restriction site serves as the annealing site for the secondary-barcode primer (oligo C, FIG. 1E). This leaves a sequence of 15 bases upstream of the ROI site and 32 bases downstream of this site that are untouched by any primer and are amplified together with the enriched ROI elements. The differences between the HBB and HBD ROI 3′-flanking sequences are used to define NGS reads as belonging to either HBB or HBD during the sequence analysis step.

An advantage of the fact that, for each donor, the HBB and HBD ROIs are processed by MEMDS simultaneously and side by side in the same reaction by the same oligos, with the consequence that the genes are only separated by their unique and small sequence differences at the computational stage, is that any mutational patterns arising in one gene and not in the other cannot be assigned to methodological artifacts. Such artifacts would have been expected to manifest themselves in both genes.

### Example 4

### In vitro Analysis of the Effects of Restriction-Site Mutations on Bsu361 Activity

To study the de novo origination rate of the HbS mutation in HBB and of the parallel A to T mutation in HBD, as well as the rates of other mutations in their vicinity, the MEMDS method was applied to the HBB and HBD genes from human-sperm DNA, exploiting the fact that codon 6 in both genes comprises a part of the recognition site for the Bsu36I restriction enzyme (RE-1) (FIG. 3). However, the enrichment of any mutation within the ROI site depends on the efficient blockage of Bsu36I digestion, and therefore the ability of all single-base substitutions to effectively block Bsu36I digestion was tested. For this purpose, the deep mutational scanning approach (Fowler DM and Fields S (2014) Nat Methods 11(8):801; Melamed D, et al (2013) RNA 19(11):1537-1551) was applied to generate a synthetic-DNA library of HBB segments carrying all possible single-base substitutions in the Bsu36I site and its flanking sequences. After incubating the library for 20 hours either with or without Bsu36I, next-generation sequencing of the full-length products that were recovered from each sample allowed the counting of each mutation variant and the calculation of the fold difference in its frequency between the two samples, which serves as a proxy to the degree of resistance to Bsu36I cleavage. In accordance with the known consensus sequence for the Bsu36I site (CCTNAGG), it was found that while the central base can tolerate any type of substitution, any single point mutation in the remaining 6 bases of the Bsu36I site is resistant to digestion (FIG. 4). A synthetic dsDNA library of HBB gene segments containing the Bsu36I-restriction site with its flanking sequences and a single point mutation per segment was divided into two and sequenced following incubation with or without Bsu36I. As Bsu36I digestion results in the depletion of sequences that are Bsu36I-sensitive, the frequency of each full-length variant in the post-Bsu36I treatment pool compared to its frequency in the pre-Bsu36I treatment pool was used to determine its degree of Bsu36I-resistance. Changes in frequencies were normalized to the change in frequency of an artificial variant with multiple changes in the Bsu36I site, which was set to 100% resistance (n=2). Bsu36I-sensitive variants are not completely depleted from the post-Bsu36I treatment pool, probably due to Bsu36I-resistant heteroduplex DNA molecules that carry a Bsu36I-sensitive sequence in one strand and a Bsu36I-resistant sequence in the second strand, formed during the PCR reaction that generated the input dsDNA library. Single-base substitutions protect from Bsu36I digestion similarly to the MEMDS-artificial ROI variant that carries multiple changes in the Bsu36I site. The degree of resistance is similar to that of a variant that carries substitutions in all of the seven bases that constitute the Bsu36I site (the same set of mutations found in ALP13, which is one of the two artificial ROIs used to determine the Bsu36I-enrichment factor). Therefore, natural single-base substitutions in Bsu36I sites are effective substrates for enrichment by Bsu36I.

### Example 5

### Generating HBB and HBD Sequence Datasets

The MEMDS protocol was applied to 7 sperm-DNA samples obtained from semen donations from donors of African ancestry (AFR1-7) and four samples from donors of European ancestry (EUR1-4) (see Table 1 for detail).

TABLE 1
General properties of sperm samples

| | Donation | Parent-1/Parent-2 | Age | Ethnic | Sperm count | % |
|---|---|---|---|---|---|---|
| Donor | year | origin | group | group | (millions/ml) | motility |
| Afr1 | 2018 | Ghanaian/Ghanaian | 23-28 | Akan | 46.1 | 45 |
| Afr2 | 2018 | Ghanaian/Ghanaian | 35-39 | Ewe | 27.6 | 38 |
| Afr3* | 2018 | Ghanaian/Ghanaian | 23-28 | Ga/Ewe | 24.1 | 43 |
| | | | | Dagbani | 20 | 40 |
| Afr4 | 2018 | Ghanaian/Ghanaian | 18-22 | Akan | 30.5 | 46 |
| Afr5 | 2019 | Ghanaian/Ghanaian | 23-28 | Akan | 51.1 | 42 |
| Afr6 | 2019 | Ghanaian/Ghanaian | 29-34 | Akan | 42.3 | 50 |
| Afr7 | 2019 | Ghanaian/Ghanaian | 23-28 | Ewe | 45.3 | 36 |
| Eur1 | 1994 | English/German | 23-28 | Caucasian | 68.0 | 50 |
| Eur2 | 2001 | English/English - | 29-34 | Caucasian | 68.7 | 40 |
| | | Scandinavian | | | | |
| Eur3 | 2005/6 | German/French- | 23-28 | Caucasian | 57.2 | 35 |
| | | Italian | | | | |
| Eur4 | 1995 | English/Norwegian | 29-34 | Caucasian | 77.8 | 43 |

*Sample contains mixed cells from two donors

As described in Examples 1 and 2, from each sample genomic DNA was aliquoted in an amount equivalent to 60-80 million sperm cells into one tube (referred to as “Bsu36I-treated”) and an amount equivalent to 5% of the cells (3-4 million sperm cells, respectively) into a second tube (referred to as “Bsu36I-untreated”). Each of the two reaction tubes was supplemented by a known amount of plasmid mixture that carries artificial Bsu36I-resistant HBB and HBD sequences. The Bsu36I-treated sample was treated with Bsu36I and HpyCH4III, and the Bsu36I-untreated sample was treated with HpyCH4III only. Except for the digestion step, the two samples were processed identically by the complete MEMDS procedure and sequenced.

Following standard quality filtration and merging of overlapping paired-end reads, reads were validated for carrying the 14-mer primary-barcode and the 5-mer secondary-barcode features, as well as the unique 5′ and 3′ sample-identifier sequences.

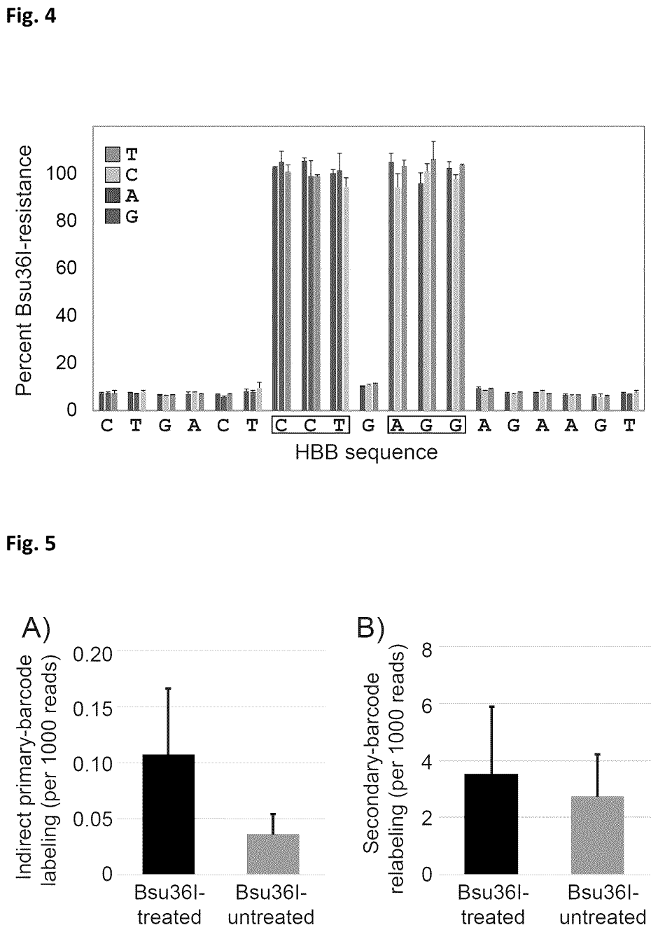

Control-guanine insertions designed to report for primary-barcode indirect labeling (see FIG. 1B, and the section termed “DNA oligos” in Materials and Methods, for oligo A features) were found to be present in ˜1/9,000 reads for the Bsu36I-treated samples and ˜1/28,000 reads for the Bsu36I-untreated samples (FIG. 5A), implying an efficient 3′inverted-dT blockage of the primary-barcode oligo. Yet, the observed difference in the fraction of reads with control-guanine insertions between the Bsu36I-treated and untreated samples suggests that the large amount of treated DNA in the former (leading, for example, to longer preparation times for some of the MEMDS step) and/or residual effects of Bsu36I digestion products may account for the elevated frequencies of indirect-labeling in the former.

After removing sequences with the control-guanine insertions, reads were sorted into separate HBB and HBD datasets based on their match to unique sequence features of each gene (see Materials and Methods and FIG. 3 for the exact sorting parameters). Consequently, each sperm sample produced four major datasets consisting of separate HBB and HBD sequencing pools for each of the Bsu36I-treated and untreated samples. Each read was then aligned against the donor's reference sequence and the presence of mutations and their types were noted per position. Next, reads were grouped into families based on their primary barcode sequences, where within each family, reads shared the same primary barcode and represented multiple copies of the same original target-DNA molecule, and each secondary barcode represented one of the ≤15 linearly amplified copies of that target molecule. Only families that passed the criteria discussed in Example 6 were selected for mutation-detection analysis.

### Example 6

### Filtering Families for Mutation Detection Analysis

Three major parameters affect the level of accuracy by which a primary-barcode family is considered as being originated from either a wild-type or a mutated target DNA molecule: a) the number of reads belonging to a primary-barcode family (i.e., family size); b) the fraction of reads in the family having the same nucleotide (either a wild type or a mutation) in a given position (mutation frequency); and c) the number of secondary barcodes in a primary-barcode family associated with either a wild-type base or a particular mutation (i.e., BC2 count).

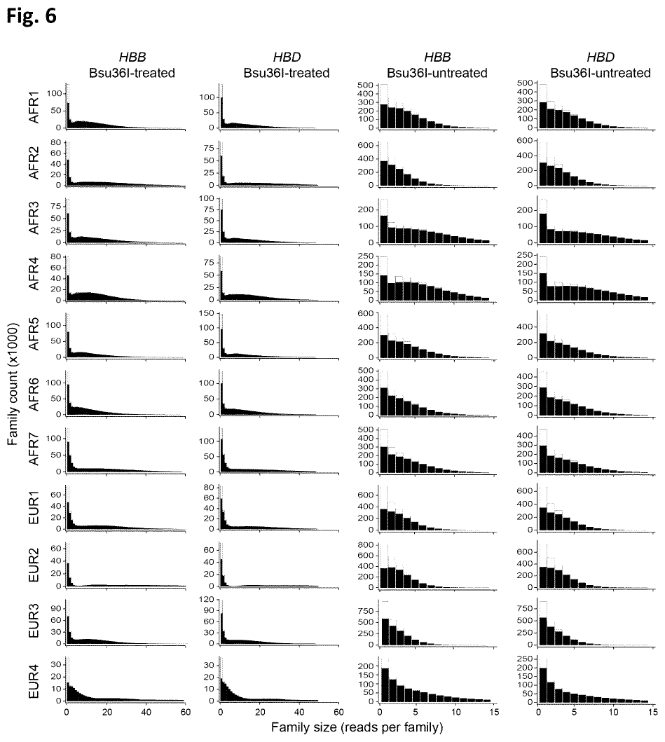

For all donors and treatments, most primary-barcode families contained multiple Nature 534(7609):693), many families were represented by single reads. It is likely that many of these single-read families represent genuine labeling events that did not accumulate enough reads during the amplification steps, and are thus excluded from analysis and are a part of the general loss of material. Additionally, we found that between 20% and more than 50% of the single-read families had a primary-barcode sequence that deviated by a Hamming distance of one from one of the primary-barcode sequences of a family with multiple reads (FIG. 6), suggesting that each of these single-read families is likely the result of a single-base error in the primary barcode of one of the reads in a multiple-reads family acquired during library preparation or NGS steps. Supporting this inference, a far smaller percentage of the primary barcodes of families with multiple reads were found to be at a Hamming distance of one away from other primary-barcode families.

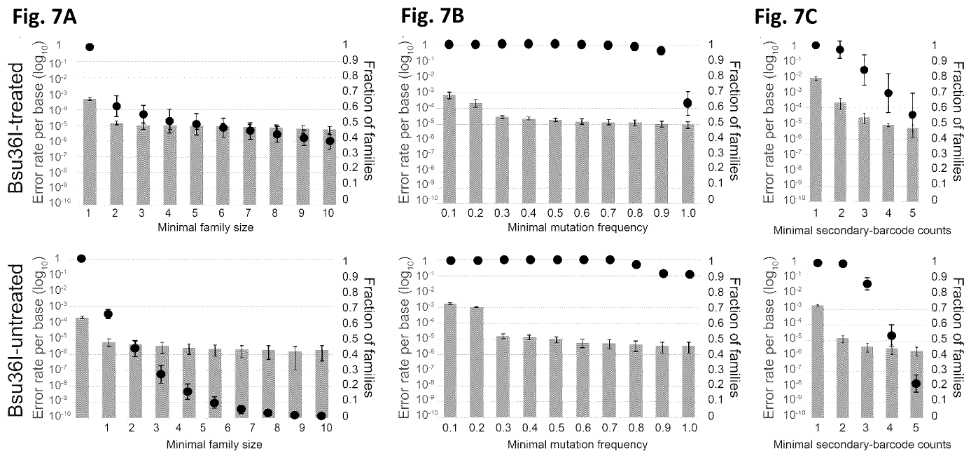

In addition to eliminating the barcode-error artifact, increasing the family size reduces the influence that sequence errors have on the final consensus sequence. Under the most stringent assumption that all the mutations appearing in sequences from the Bsu36I-untreated samples and in the ROI-flanking sequences from the Bsu36I-treated samples are due to NGS or PCR errors, gradually increasing the family-size cutoff reduced the acceptance rate of these false positive mutations for both samples (FIG. 7A). A minimum required family size of four reads was selected for further mutation detection analysis, as increasing the family size cutoff beyond 4 reads did not noticeably improve mutation detection accuracy but continued to reduce the number of recovered families (FIG. 7A).

Increasing the mutation frequency cutoff, i.e., the minimal fraction of reads in a family carrying a particular nucleotide in a particular position allowing us to accept that nucleotide, reduced the fraction of false positive mutant families already when using low cutoff values, suggesting that the source of these mutations are late PCR errors or NGS errors that appear in small fractions within families (FIG. 7B). A mutation-frequency cutoff of 0.7 was selected (i.e., at least 70% of the family members carried either a wild-type base or a particular mutation at a given position), which provided a good balance between the number of mutations that were filtered out and the number of recovered families.

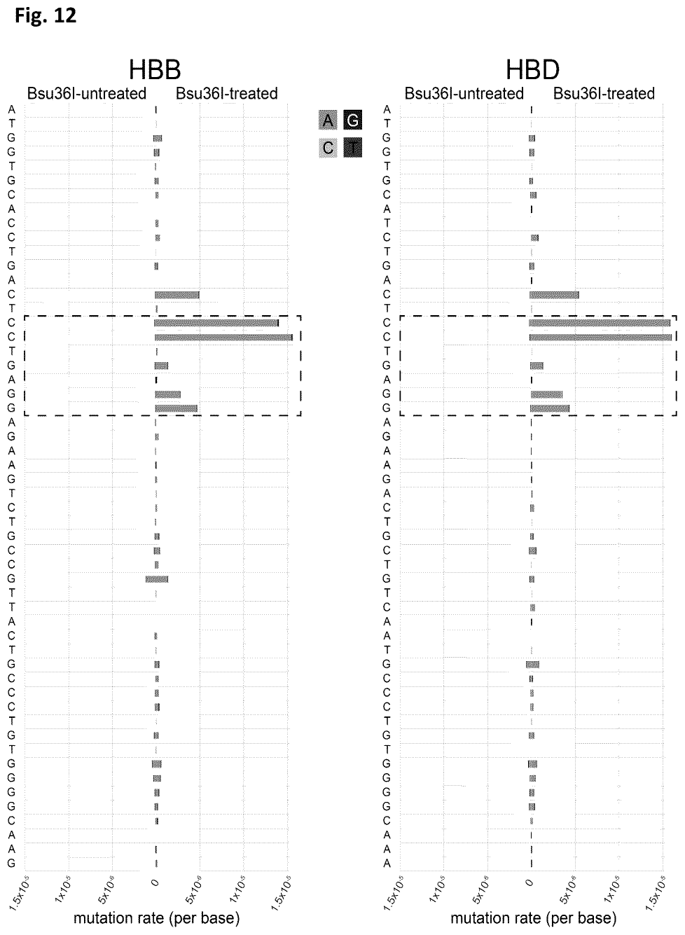

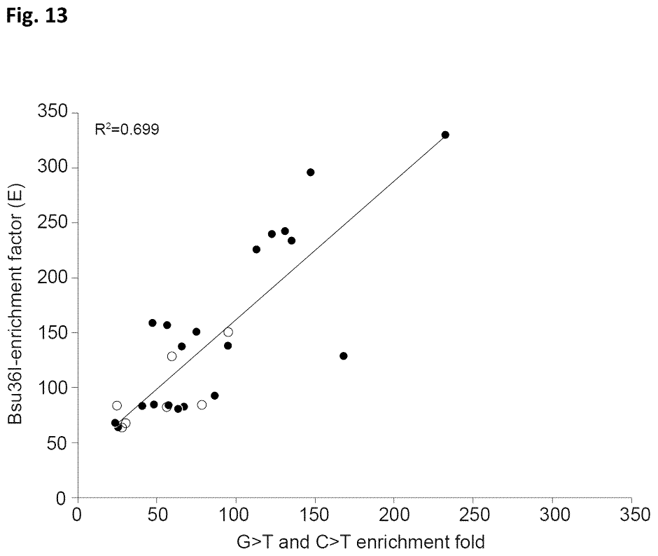

Chimeric HBB/HBD markers (HBB 9C to T and HBD 9T to C substitutions) were not included in the mutation count (Example 9), nor was the ROI-flanking sequence mutation 14C to A that was found to be enriched by Bsu36I digestion (FIG. 12 and Example 8).

For each family, the number of unique secondary barcodes that were added after the linear amplification step and before the PCR amplification step corresponds to the number of unique linearly amplified copies of the original DNA molecule. Therefore, requiring multiple secondary barcodes allows to reduce the error rate by ensuring that reads from distinct linear amplification events are used in the analysis. For the families with the highest read counts, it was found that usually 4-5 of the unique secondary barcodes were more frequent than the remaining secondary barcodes, suggesting that while some of the linearly amplified copies of each ROI were PCR amplified more efficiently than others their repertoire was diverse enough and not over dominated by a single linearly amplified copy. Negligible amounts of families with more than 15 unique secondary barcodes, which matches the maximal number of linearly amplified copies and supports the authenticity of these barcodes were found. The control for secondary barcode relabeling suggests that such an event occurs once every 250-350 reads (FIG. 5B). Since both the originally labeled and the erroneously relabeled copies need to be sampled and included in the same family for their secondary barcodes to be miscounted twice, the negative effect of this event should be even smaller. Limiting mutation calling by requiring a minimum of two secondary barcodes associated with a particular nucleotide in a particular position as a condition for that nucleotide to be accepted (whether it is a wild type or a mutation), in addition to the family size cutoff, improved accuracy with a minimal effect on the number of recovered families (FIG. 7C). Thus, besides the major contribution of mutation enrichment by restriction-enzyme digestion to mutation detection accuracy, setting up a secondary-barcode count cutoff as a regular part of the MEMDS procedure adds further precision in mutation calling in comparison to the MDS method (Jee J, et al. (2016) Nature 534(7609):693).

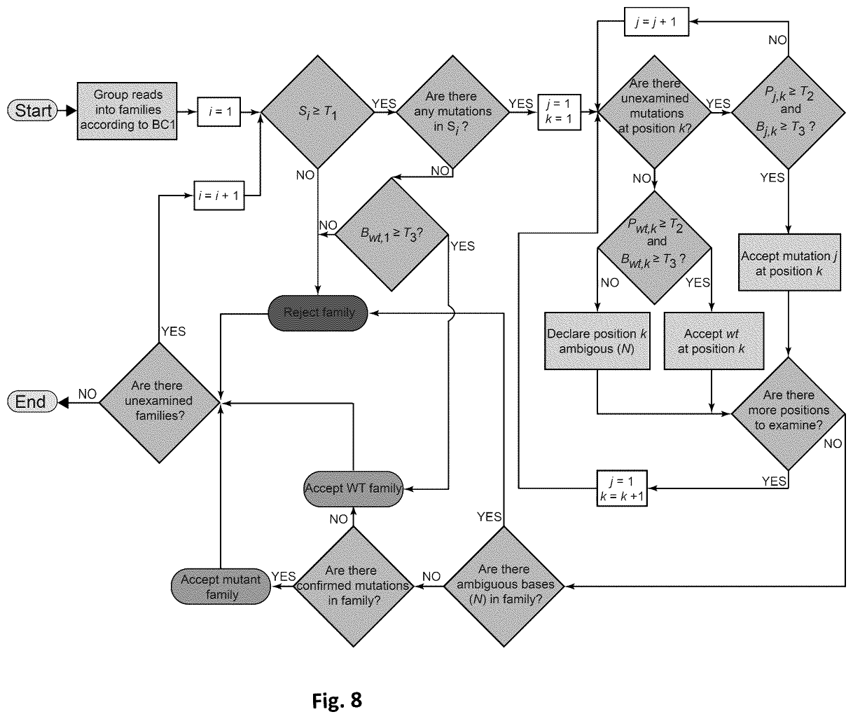

Based on the above considerations the following combined threshold criteria were selected: primary-barcode families with at least four reads, a minimal within-family mutation frequency cutoff of 70%, and the association of at least two secondary barcodes with each base. The flowchart of the algorithm that was developed for base calling is provided in FIG. 8. As shown in FIG. 8, The workflow describes the computational analysis from the point of grouping reads into families by their shared primary barcodes, where each family represents a single target-DNA molecule, to the characterization of each family by its mutations that pass the combined cutoff criteria if they exist. As indicated above, these criteria include a minimal family size of four reads (T1), a mutation frequency cutoff of at least 0.7 (T2) and the association of the specific mutation called with at least two secondary barcodes (T3). For any given mutated position, in the case of failure to pass the combined cutoff criteria, the wild-type base is tested by the same conditions to validate its authenticity in an unbiased manner. If both the mutation and the wild-type base fail to meet the cutoff criteria, the base identity at that position is declared ambiguous (N), and the family is rejected.

Importantly, the same criteria were used for calling a wild-type family or a mutant family, thus eliminating any computational bias that would have been associated with different treatments of wild type and mutant and could affect the calculation of de novo mutation rates. In those rare events where neither the wild-type nucleotide nor a particular mutation in a ≥4-read family meet the mutation-frequency cutoff or the secondary-barcode count cutoff conditions at a certain position, the nucleotide identity at that position is declared ambiguous and the family is rejected from further analysis.

The numbers of families that were rejected or approved by these three cutoff criteria are shown for each library in Table 2. HBB and HBD families that shared the same primary-barcode sequences, which point to HBB/HBD chimeric artifacts that were generated during library amplification were also removed from further analysis.

TABLE 2
Numbers of rejected and approved gene families following
filtration by the combined cutoff criteria:

| | | | | | Rejected | Rejected | | Approved |
|---|---|---|---|---|---|---|---|---|
| | | | | | due to WT | due to | Rejected | by the |
| | | | | Rejected | secondary | shared | due to | combined |
| | | | Total | due to low | Barcode | primary | ambiguous | cutoff |
| Donor | Treatment | Gene | Families¹ | Counts² | failure³ | barcodes⁴ | bases (N)⁵ | criteria |
| AFRI | Bsu36I- | HBB | 577,003 | 174,577 | 33,474 | 2,865 | 6,985 | 359,102 |
| | Treated | HBD | 492,594 | 189,270 | 31,969 | 2,865 | 7,390 | 261,110 |
| | Bsu36I- | HBB | 1,876,444 | 1,154,913 | 5,796 | 4,569 | 3,052 | 708,114 |
| | untreated | HBD | 1,698,297 | 1,035,754 | 6,657 | 4,569 | 3,222 | 648,095 |
| AFR2 | Bsu36I- | HBB | 326,152 | 105,610 | 12,819 | 2,326 | 2,910 | 202,487 |
| | Treated | HBD | 295,836 | 115,683 | 11,330 | 2,326 | 2,862 | 163,635 |
| | Bsu36I- | HBB | 1,784,865 | 1,374,897 | 6,232 | 2,583 | 2,136 | 399,017 |
| | Untreated | HBD | 1,753,711 | 1,259,296 | 5,899 | 2,583 | 2,344 | 483,589 |
| AFR3 | Bsu36I- | HBB | 386,700 | 126,712 | 16,860 | 3,104 | 8,990 | 231,034 |
| | Treated | HBD | 349,216 | 137,830 | 18,259 | 3,104 | 5,435 | 184,588 |
| | Bsu36I- | HBB | 1,148,389 | 497,865 | 9,729 | 3,725 | 5,900 | 631,170 |
| | untreated | HBD | 1,051,314 | 464,336 | 10,926 | 3,725 | 3,520 | 568,807 |
| AFR4 | Bsu36I- | HBB | 426,278 | 107,727 | 19,883 | 987 | 3,122 | 294,559 |
| | Treated | HBD | 372,518 | 113,201 | 9,855 | 987 | 14,838 | 233,637 |
| | Bsu36I- | HBB | 1,264,724 | 521,564 | 21,801 | 4,290 | 4,563 | 712,506 |
| | untreated | HBD | 1,120,079 | 450,195 | 11,035 | 4,290 | 22,693 | 631,866 |
| AFR5 | Bsu36I- | HBB | 463,684 | 185,935 | 15,604 | 4,054 | 29,396 | 228,695 |
| | Treated | HBD | 420,559 | 196,822 | 28,311 | 4,054 | 9,221 | 182,151 |
| | Bsu36I- | HBB | 1,891,157 | 1,174,471 | 7,638 | 4,031 | 19,437 | 685,580 |
| | untreated | HBD | 1,746,131 | 1,103,189 | 14,760 | 4,031 | 5,622 | 618,529 |
| AFR6 | Bsu36I- | HBB | 573,258 | 208,356 | 29,491 | 3,613 | 7,612 | 324,186 |
| | Treated | HBD | 531,781 | 200,664 | 22,825 | 3,613 | 5,782 | 298,897 |
| | Bsu36I- | HBB | 1,575,956 | 1,014,909 | 3,497 | 3,292 | 3,864 | 550,394 |
| | untreated | HBD | 1,481,666 | 903,810 | 3,176 | 3,292 | 3,305 | 568,083 |
| AFR7 | Bsu36I- | HBB | 574,043 | 212,633 | 10,969 | 32,028 | 3,850 | 314,563 |
| | Treated | HBD | 541,285 | 234,389 | 11,208 | 32,028 | 3,758 | 259,902 |
| | Bsu36I- | HBB | 1,699,015 | 1,043,765 | 10,971 | 4,513 | 5,811 | 633,955 |
| | untreated | HBD | 1,555,197 | 932,933 | 9,599 | 4,513 | 5,004 | 603,148 |
| EUR1 | Bsu36I- | HBB | 331,608 | 126,256 | 3,737 | 19,141 | 1,229 | 181,245 |
| | Treated | HBD | 311,712 | 139,262 | 3,252 | 19,141 | 1,422 | 148,635 |
| | Bsu36I- | HBB | 2,144,051 | 1,589,762 | 2,462 | 4,199 | 2,161 | 545,467 |
| | untreated | HBD | 1,936,890 | 1,388,844 | 2,327 | 4,199 | 2,317 | 539,203 |
| EUR2 | Bsu36I- | HBB | 228,770 | 89,463 | 7,512 | 4,692 | 2,623 | 124,480 |
| | Treated | HBD | 210,577 | 97,700 | 6,413 | 4,692 | 2,734 | 99,038 |
| | Bsu36I- | HBB | 2,392,244 | 1,827,090 | 3,029 | 4,056 | 1,565 | 556,504 |
| | untreated | HBD | 2,172,098 | 1,619,546 | 3,392 | 4,056 | 1,725 | 543,379 |
| EUR3 | Bsu36I- | HBB | 418,458 | 161,339 | 3236 | 7,226 | 7,428 | 239,229 |
| | Treated | HBD | 393,302 | 172,746 | 7,036 | 7,226 | 3,384 | 202,910 |
| | Bsu36I- | HBB | 2,335,295 | 1,888,649 | 422 | 2,698 | 11,427 | 432,099 |
| | untreated | HBD | 2,143,059 | 1,712,093 | 778 | 2,698 | 3,942 | 423,548 |
| EUR4 | Bsu36I- | HBB | 144,833 | 43,533 | 20,359 | 36 | 13,548 | 67,357 |
| | Treated | HBD | 122,042 | 38,710 | 18,175 | 36 | 11,712 | 53,409 |
| | Bsu36I- | HBB | 731,846 | 242,928 | 122,442 | 1,102 | 26,355 | 339,019 |
| | untreated | HBD | 649,808 | 215,711 | 98,264 | 1,102 | 19,597 | 315,134 |

¹Total number of families subjected to the combined cutoff criteria.
²Families failing to meet the family size cutoff of ≥4.
³Wild-type families with at least 4 reads that fail to meet a secondary-barcode count cutoff of ≥2.
⁴Families that share their primary-barcode sequences between HBB and HBD genes of the same donor and treatment (chimeric artifacts).
⁵Mutation-containing families that fail to meet a mutation-frequency cutoff of ≥0:7 or a secondary-barcode count cutoff of ≥2 for either a mutation or a wild-type base at least in one position in sequence.
6. The combined cutoff criteria include a family size cutoff of ≥4, a mutation frequency cutoff of ≥0:7 and a secondary-barcode count cutoff of ≥2.
The numbers of rejected and approved families sum up to the total number of families.

### Example 7

### MEMDS Performance Measures: Enrichment Factors, Numbers of Genomes Scanned, Mutation Recovery Rate and Error Rate

For determining the origination rate of a particular mutation at a particular site, one must divide the number of sampled target sequences carrying that mutation by the total number of sampled target sequences. For the Bsu36I-untreated samples, the total number of target sequences sampled is derived solely from the number of families that are present in the sequencing output and that have passed the combined cutoff criteria. For the Bsu36I-treated samples, however, the total number of target sequences sampled (the number of genomes scanned by MEMDS) must also include the number of target sequences that have been eliminated due to Bsu36I digestion. This number is derived using the method described in Examples 1 and 2. To recapitulate, the ratio between the number of artificial Bsu36I-resistant families and the number of wild-type families that result from applying the MEMDS procedure to the input mixture of the Bsu36I-treated sample is divided by the analogous ratio from the untreated sample, while correcting for the different volumes drawn for practical considerations from different source tubes, to obtain the Bsu36I-enrichment factor. The number of scanned wild-type target sequences (i.e., the number of target sequences that had been removed by Bsu36I digestion plus the number of target sequences that escaped Bsu36I digestion and formed wild-type families that passed the cutoff criteria) is then calculated by multiplying the number of wild-type families that passed the cutoff criteria by the Bsu36I-enrichment factor.

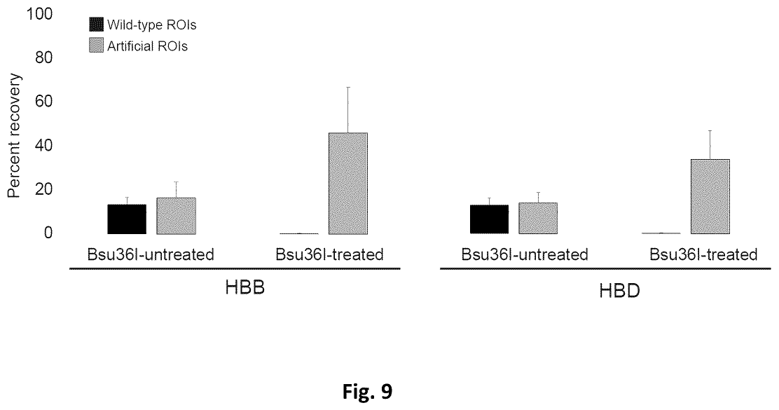

On average, about 13% of the input HBB and HBD wild-type ROIs were recovered in the Bsu36I-untreated samples (FIG. 9). A similar recovery rate of 15% was observed for the artificial ROIs in these samples, suggesting that both the genomic and the plasmid variants are processed similarly by this MEMDS workflow. Notably, the relatively low recovery rate of both target molecules is likely due to the overload input DNA, as no restriction enzyme-based depletion of wild-type ROI sequences takes place in these samples. However, these recovery rates satisfy the main purpose of the Bsu36I-untreated samples, which is to set the artificial ROI/genomic ROI ratio in the absence of Bsu36I enrichment (see Examples 1 and 2).

Percent recoveries of the WT and artificial target molecules were calculated using the ratios between the obtained number of families of each type and the estimated amounts of input families derived from their DNA concentration measurements.

Following Bsu36I treatment, the wild-type ROI levels dropped to an average of ˜0.25% of their input levels, while the artificial ROI levels were at an average of ˜40% of their input levels, with HBB recovery being slightly higher than the recovery of HBD (FIG. 9). Given that point mutations at the ROI block Bsu36I digestion and are enriched similarly to the artificial sequences (FIG. 4), this percent recovery of the artificial ROIs suggests that at least a third of Bsu36I-resistant mutations that were present in the input sperm DNA were recovered in the Bsu36I-treated samples by the MEMDS procedure.

For each donor, the Bsu36I enrichment factors obtained from the artificial ROI/genomic ROI ratios showed a high degree of consistency between HBB and HBD, which reflects a similar activity of Bsu36I on both genes (Table 3). However, these enrichment factors displayed some variation across donors, ranging from a 64-fold enrichment to a 340-fold enrichment, likely associated with either differences in Bsu36I activity due to batch effects or differences in the integrity of the sperm DNA (namely, the fraction of HBB and HBD ROI segments that were in a double-stranded state). In particular, the average enrichment factor of the four European samples (278.0±47.8) was about 2.6-fold higher than the average enrichment factor of the seven African samples (107.3±35.0). This difference in the average enrichment factor, which represents a Bsu36I digestion of 99.6% of the European HBB and HBD ROI sequences compared to 99.1% of the African ROI sequences, could arise due to the differences in semen composition between the two groups of donors that affect the double-strand state of the genomic DNA during its extraction. The enrichments of the highly frequent G to T and C to T substitutions in the target DNA strand (C to A and G to A in the sequenced strand, respectively) at the ROIs were found to exhibit differences between the eleven donors that followed the same direction as the differences between the Bsu36I-enrichment factors, which further supports the Bsu36I-enrichment-factor calculation according to the invention (see Example 8). Thus, given an average enrichment factor of ˜170-fold, the enrichment step of MEMDS alone boosts both the sequence coverage in the search for the target de novo mutations and the accuracy of mutation-detection by more than two orders of magnitude in comparison to the mutation rate in the Bsu36I-untreated samples.

TABLE 3
Values for the calculation of Bsu36I-enrichment factors and numbers of scanned target DNA sequences.

| | | | | | | | | | Plasmid | | |
|---|---|---|---|---|---|---|---|---|---|---|---|
| | | | | | | | | | families | | Scanned |
| | | Donor DNA | | Plasmid DNA | | | WT families | | passing | Enrichment | Target |
| Donor | Treatment | Volume taken | | Volume taken | | Gene | passing cutoff¹ | | cutoff² | factor³ | sequences⁴ |
| AFR1 | Bsu36I- | 700 μl | (V_{Se}) | 20 μl | (V_{Re}) | HBB | 350,937 | (S_{g}^{f}) | 3,657; 3,899 (R_{g}^{f}) | 63.60 | 22,319,593 |
| | treated | | | | | HBD | 254,507 | (S_{g}^{f}) | 2,939; 3,158 (R_{g}^{f}) | 67.04 | 17,062,149 |
| | Bsu36I- | 35 μl | (V_{Se}) | 120 μl | (V_{Re}) | HBB | 680,429 | (S_{g}^{f}) | 12,275; 15,367 (R_{g}^{f}) | 1.00 | 880,429 |
| | untreated | | | | | HBD | 621,399 | (S_{g}^{f}) | 12,156; 14,492 (R_{g}^{f}) | 1.00 | 621,399 |
| AFR2 | Bsu36I- | 338 μl | (V_{Se}) | 20 μl | (V_{Re}) | HBB | 194,704 | (S_{g}^{f}) | 3,483; 3,803 (R_{g}^{f}) | 156.56 | 30,872,266 |
| | treated | | | | | HBD | 157,003 | (S_{g}^{f}) | 3,057; 3,200 (R_{g}^{f}) | 156.05 | 24,500,318 |
| | Bsu36I- | 16.9 μl | (V_{Se}) | 120 μl | (V_{Re}) | HBB | 388,011 | (S_{g}^{f}) | 4,682; 6,307 (R_{g}^{f}) | 1.00 | 388,011 |
| | untreated | | | | | HBD | 469,182 | (S_{g}^{f}) | 6,077; 8,302 (R_{g}^{f}) | 1.00 | 469,182 |
| AFR3 | Bsu36I- | 744 μl | (V_{Se}) | 20 μl | (V_{Re}) | HBB | 194,315 | (S_{g}^{f}) | 4,179; 3917 (R_{g}^{f}) | 150.09 | 29,164,431 |
| | treated | | | | | HBD | 177,422 | (S_{g}^{f}) | 3,463; 3,227 (R_{g}^{f}) | 128.13 | 22,696,345 |
| | Bsu36I- | 37.2 μl | (V_{Se}) | 120 μl | (V_{Re}) | HBB | 610,803 | (S_{g}^{f}) | 10,760; 9,887 (R_{g}^{f}) | 1.00 | 610,803 |
| | untreated | | | | | HBD | 549,390 | (S_{g}^{f}) | 10,267; 9,135 (R_{g}^{f}) | 1.00 | 549,390 |
| AFR4 | Bsu36I- | 720 μl | (V_{Se}) | 20 μl | (V_{Re}) | HBB | 286,939 | (S_{g}^{f}) | 3,561; 3,341 (R_{g}^{f}) | 82.85 | 23,773,196 |
| | treated | | | | | HBD | 227,197 | (S_{g}^{f}) | 2,931; 2,908 (R_{g}^{f}) | 83.65 | 19,005,979 |
| | Bsu36I- | 36 μl | (V_{Se}) | 120 μl | (V_{Re}) | HBB | 688,478 | (S_{g}^{f}) | 12,538; 11,448 (R_{g}^{f}) | 1.00 | 688,478 |
| | untreated | | | | | HBD | 609,365 | (S_{g}^{f}) | 11,616; 10,849 (R_{g}^{f}) | 1.00 | 609,365 |
| AFR5 | Bsu36I- | 600 μl | (V_{Se}) | 20 μl | (V_{Re}) | HBB | 211,556 | (S_{g}^{f}) | 9,274; 7,321 (R_{g}^{f}) | 91.87 | 19,436,031 |
| | treated | | | | | HBD | 172,778 | (S_{g}^{f}) | 5,007; 3,830 (R_{g}^{f}) | 83.92 | 14,500,198 |
| | Bsu36I- | 30 μl | (V_{Se}) | 120 μl | (V_{Re}) | HBB | 621,848 | (S_{g}^{f}) | 37,351; 26,363 (R_{g}^{f}) | 1.00 | 621,848 |
| | untreated | | | | | HBD | 576,349 | (S_{g}^{f}) | 25,761; 16,389 (R_{g}^{f}) | 1.00 | 576,349 |
| AFR6 | Bsu36I- | 320 μl | (V_{Se}) | 20 μl | ((V_{Re})) | HBB | 305,108 | (S_{g}^{f}) | 9,731; 8,726 (R_{g}^{f}) | 82.10 | 25,048,983 |
| | treated | | | | | HBD | 285,210 | (S_{g}^{f}) | 7,152; 5,863 (R_{g}^{f}) | 80.18 | 22,867,577 |
| | Bsu36I- | 16 μl | (V_{Se}) | 120 μl | (V_{Re}) | HBB | 505,664 | (S_{g}^{f}) | 25,122; 19,589 (R_{g}^{f}) | 1.00 | 505,664 |
| | untreated | | | | | HBD | 531,747 | (S_{g}^{f}) | 20,863; 15,454 (R_{g}^{f}) | 1.00 | 531,747 |
| AFR7 | Bsu36I- | 360 μl | (V_{Se}) | 20 μl | (V_{Re}) | HBB | 284,362 | (S_{g}^{f}) | 15,848; 13,647 (R_{g}^{f}) | 137.55 | 39,114,652 |
| | treated | | | | | HBD | 239,630 | (S_{g}^{f}) | 10,693; 8,789 (R_{g}^{f}) | 136.97 | 32,848,562 |
| | Bsu36I- | 18 μl | (V_{Se}) | 120 μl | (V_{Re}) | HBB | 581,338 | (S_{g}^{f}) | 29,950; 22,654 (R_{g}^{f}) | 1.00 | 581,338 |
| | untreated | | | | | HBD | 563,058 | (S_{g}^{f}) | 22,790; 17,283 (R_{g}^{f}) | 1.00 | 563,058 |
| EUR1 | Bsu36I- | 592 μl | (V_{Se}) | 20 μl | (V_{Re}) | HBB | 173,119 | (S_{g}^{f}) | 3,696; 4,081 (R_{g}^{f}) | 225.50 | 39,038,335 |
| | treated | | | | | HBD | 141,078 | (S_{g}^{f}) | 3,460; 3,731 (R_{g}^{f}) | 239.46 | 33,782,538 |
| | Bsu36I- | 29.6 μl | (V_{Se}) | 120 μl | (V_{Re}) | HBB | 532,724 | (S_{g}^{f}) | 5,431; 7,304 (R_{g}^{f}) | 1.00 | 532,724 |
| | untreated | | | | | HBD | 525,762 | (S_{g}^{f}) | 5,951; 7,479 (R_{g}^{f}) | 1.00 | 525,762 |
| EUR2 | Bsu36I- | 780 μl | (V_{Se}) | 20 μl | (V_{Re}) | HBB | 118,015 | (S_{g}^{f}) | 3,020; 3,231 (R_{g}^{f}) | 329.27 | 38,858,799 |
| | treated | | | | | HBD | 93,487 | (S_{g}^{f}) | 2,669; 2,712 (R_{g}^{f}) | 340.41 | 31,823,910 |
| | Bsu36I- | 39 μl | (V_{Se}) | 120 μl | (V_{Re}) | HBB | 545,961 | (S_{g}^{f}) | 4,499; 6,040 (R_{g}^{f}) | 1.00 | 545,961 |
| | untreated | | | | | HBD | 532,567 | (S_{g}^{f}) | 4,788; 6,018 (R_{g}^{f}) | 1.00 | 532,567 |
| EUR3 | Bsu36I- | 440 μl | (V_{Se}) | 20 μl | (V_{Re}) | HBB | 210,009 | (S_{g}^{f}) | 15,676; 13,036 (R_{g}^{f}) | 233.12 | 48,956,676 |
| | treated | | | | | HBD | 183,054 | (S_{g}^{f}) | 10,589; 8,828 (R_{g}^{f}) | 242.05 | 44,308,361 |
| | Bsu36I- | 22 μl | (V_{Se}) | 120 μl | (V_{Re}) | HBB | 403,681 | (S_{g}^{f}) | 17,836; 10,574 (R_{g}^{f}) | 1.00 | 403,681 |
| | untreated | | | | | HBD | 402,381 | (S_{g}^{f}) | 13,357; 7,803 (R_{g}^{f}) | 1.00 | 402,381 |
| EUR4 | Bsu36I- | 720 μl | (V_{Se}) | 20 μl | (V_{Re}) | HBB | 55,898 | (S_{g}^{f}) | 6,135; 5,215 (R_{g}^{f}) | 295.65 | 16,526,517 |
| | treated | | | | | HBD | 45,832 | (S_{g}^{f}) | 4,077; 3403 (R_{g}^{f}) | 318.23 | 14,585,464 |
| | Bsu36I- | 36 μl | (V_{Se}) | 120 μl | (V_{Re}) | HBB | 313,203 | (S_{g}^{f}) | 13,410; 12,402 (R_{g}^{f}) | 1.00 | 313,203 |
| | untreated | | | | | HBD | 296,860 | (S_{g}^{f}) | 9,937; 8332 (R_{g}^{f}) | 1.00 | 296,860 |

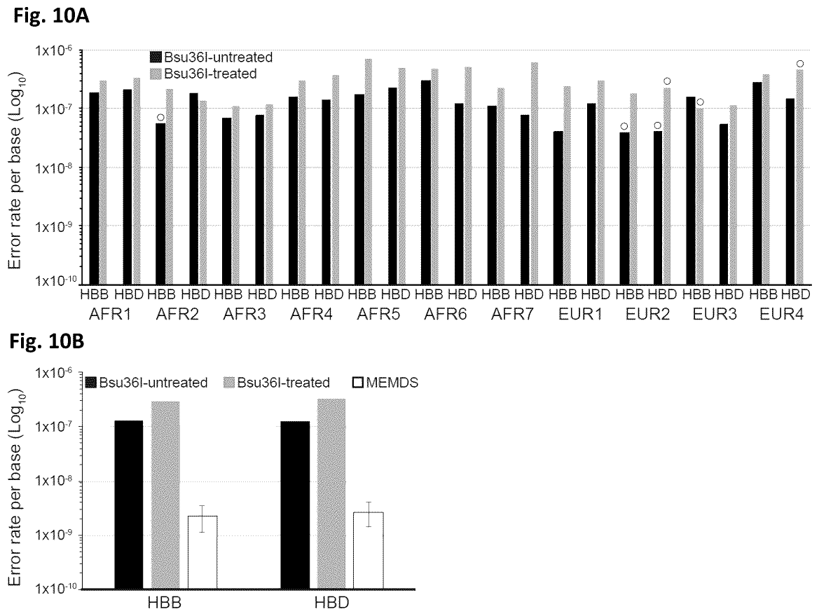

Based on the calculated enrichment factors, the total number of wild-type HBB and HBD target sequences that were screened by Bsu36I (i.e., in the Bsu36I-treated samples) reaches about 300 million for each gene (Table 3). These numbers represent an average recovery rate of slightly more than 35% for wild-type target sequences, which is highly similar to the recovery rate of the artificial ROIs, further supporting the similar processing of both the plasmid and the genomic variants by the MEMDS procedure. To calculate the MEMDS error rate, all G to T, C to T and C to A mutations in the target-DNA strand were removed from analysis due to the reasons discussed in Example 8. Under the most stringent assumption that all remaining mutations found at the ROI and ROI flanking sequences in the Bsu36I-untreated samples and at the ROI-flanking sequence in the Bsu36I-treated samples arose due to PCR or NGS errors, the total per-base error rates for the Bsu36I-untreated and treated samples across all genes and donors in these unenriched sequences was 1.3×10⁻⁷ and 3.1×10⁻⁷ per base, respectively (FIG. 10A) (error rate calculation for an individual gene from a single donor would be less accurate due to low counts of non G to T, C to T and C to A mutations in these sequences). This 2.4-fold difference between the two error rates could result from a secondary influence of the large amount of treated DNA in the former, residual effects of Bsu36I-digestion products, or from moderate enrichment of mutations outside the Bsu36I-recognition site that may display weak inhibitory effects on Bsu36I. Taking a conservative approach, the error rate at the ROI-flanking sequences in the Bsu36I-treated samples was selected as the base line for the calculation of the MEMDS error rate. The MEMDS error rates for the 6 bases that constitute the ROI were calculated for each donor by dividing the total error rate achieved for the ROI-flanking sequences of the Bsu36I-treated samples by the relevant Bsu36I-enrichment factor.

Therefore, to determine the MEMDS error rate, namely the error rate within the 6 bases of the ROI site in the Bsu36I-treated samples, for each donor and each of the two genes, the error rate obtained for the ROI-flanking sequences of HBB and HBD in the Bsu36I-treated samples (2.9×10⁻⁷ and 3.3×10⁻⁷, respectively) was divided by their matching enrichment factors, reaching an average per-base error rate of 2.3×10⁻⁹ (±1.2×10⁻⁹) and 2.6×10⁻⁹ (±1.4×10⁻⁹) for HBB and HBD, respectively, and an average of 2.5×10⁻⁹ (±1.3×10⁻⁹) for both genes (FIG. 10B). Together with MEMDS's substantial reduction in sequencing cost, this error rate enables the identification of specific de-novo mutations at particular bases of interest that originate at rates even lower than the whole genome average mutation rate in humans.

### Example 8

### G to T, C to T and C to A Mutations

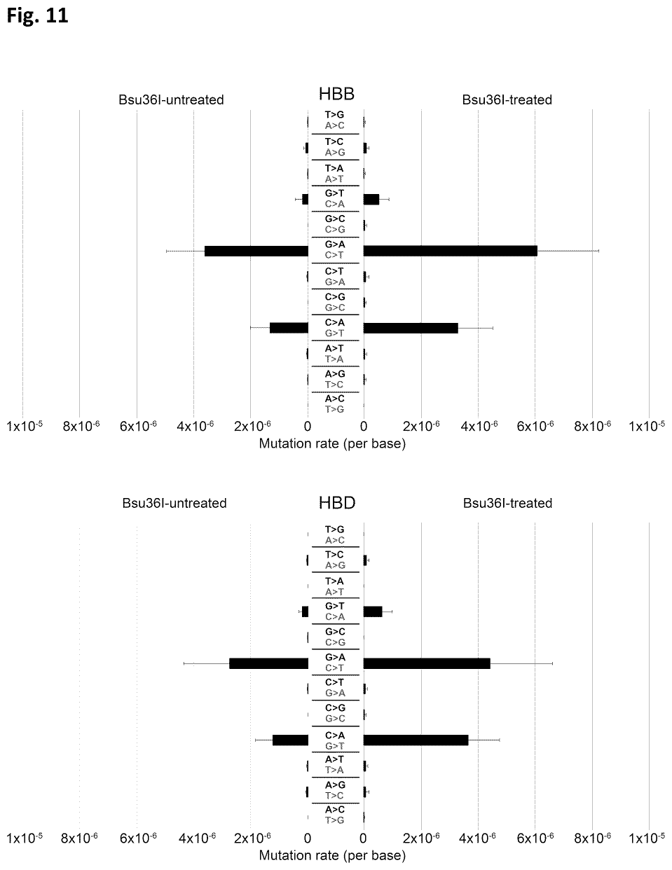

With respect to the ROI-flanking sequences both the Bsu36I-treated and the Bsu36I-untreated samples displayed similar mutation patterns (FIGS. 11 and 12). In FIG. 11, average point mutation frequencies were calculated for the 47 base sequence that includes the ROI-flanking sequences of the Bsu36I treated and untreated samples. The chimeric HBB/HBD markers (HBB 9C to T and HBD 9T to C substitutions) were not included in the mutation count (FIG. 12 and Example 9), nor was the ROI-flanking sequence mutation 14C to A that was found to be enriched by Bsu36I digestion (FIG. 12 and Example 8). Pearson's correlation coefficients of 0.934 for HBB and 0.878 for HBD, were calculated for the mutations depicted in FIG. 12.

Inspecting this mutational spectrum revealed high frequencies of single-base substitutions of three types, two of which were C to A and G to A, with average rates of ˜2.4×10⁻⁶ (±1.4×10⁻⁶) and ˜4:2×10⁻⁶ (±2.2×10⁻⁹), resp., across both genes, treatments, and donors. Since the consensus sequences are composed of reads of HBB and HBD at the sense orientation, these mutations are the reciprocals of the G to T and C to T mutations, respectively, that were present in the target, antisense DNA strand.

A major cause of G to T mutations is DNA damage occurring both endogenously under normal metabolic conditions and during DNA extraction and NGS preparation procedures. Reactive oxygen species (ROS) that arise as by-products of normal aerobic metabolism or due to the high temperatures used during DNA purification and PCR amplification steps can damage the genomic DNA by oxidizing guanine to 8-oxoguanine (8-oxoG), which in turn can pair up with adenine (8-oxoG:A) and promote a G:C to T:A mutation. C to T mutations occur either naturally or in vitro by heat-induced hydrolytic deamination of either cytosine or 5-methylcytosine (5-meC) that generate uracil or thymine, respectively. These bases can then pair up with adenine and facilitate a C:G to T:A transition.

In the HBB and HBD target antisense strands, G to T substitutions constituted ˜27% and ˜35% of the mutations found across Bsu36I-untreated and treated samples, respectively, and C to T substitutions were ˜67% and ˜56% of the mutations across the same samples, respectively. Compared to these high rates, the rates of the reciprocal substitutions in the same strands were found to be much lower: 4% and 6 for C to A, and less than 0.5% for G to A for each treatment (FIG. 11). As in previous studies that used one of the two DNA strands to explore de novo mutations, these imbalanced frequencies are taken to indicate that the formation of 8-oxoG and deaminated cytosines (or 5-meC) occurs in the DNA either in vivo or in vitro before the library amplification step, while the subsequent completion to full G:C to T:A and C:G to T:A substitutions, respectively, occurs during library amplification and not before then. Such DNA damages occurring in vitro before the library amplification step and/or representing the snapshot image of a disrupted ongoing process of base-damage and repair in vivo could result in mutational reads only when they occur in the target, antisense strand and not when they occur in the sequenced, sense strand, explaining the target-strand imbalance mentioned above.

Examining the mutation distribution along HBB and HBD sequences across all donors and treatments (FIG. 12) reveals that both the G to T and C to T substitutions in the target, antisense strand, were enriched at the ROI site, suggesting that both these types of DNA damages were formed at the target strand either in vivo or in vitro prior to the enzymatic digestion step (FIG. 1A) and conferred Bsu36I resistance. Indeed, 8-oxoG modifications placed at restriction sites or near them have been shown to interfere with the activity of multiple restriction enzymes. Similarly, generation of T:G or U:G mismatches that result from the deamination of cytosine or 5-meC have also been shown to inhibit enzymatic digestion.

Importantly, while the frequency of the C to A mutation (the reciprocal of G to T) in the target strand was much lower than that of the G to T mutation, it was still noticeably higher than those of all other point mutations besides G to T and C to T, with an average rate of ˜3.9×10⁻⁷ (FIG. 11). This observation implies that some of the guanine-oxidative damages could have affected the DNA sense strand (the non-target strand) early enough during library amplification and thus were able to produce mutations that were approved by the combined cutoff criteria.

The high frequency of the G to T and C to T substitutions in the target, antisense strand at the ROI site in the Bsu36I-treated and untreated samples allowed to calculate their enrichment fold in a manner entirely independent from the enrichment-fold calculation based on the artificial sequences described in Examples 1 and 2 (albeit more limited and less accurate than the latter as these mutations were either too infrequent or absent from the ROI of every Bsu36I-untreated sample). The enrichment of these substitutions was found to follow the same trend as the Bsu36I-enrichment factors, i.e., samples with higher enrichment factor values calculated from the artificial sequences showed increased G to T and C to T enrichments at the ROI site in comparison to samples with lower enrichment factor values (FIG. 14). In absolute terms, G to T and C to T enrichment values were lower than their matching enrichment factors calculated from the artificial sequences, likely due to 8-oxoG damages providing only incomplete resistance to Bsu36I digestion and/or continuous DNA damage occurring after the restriction enzyme digestion and affecting uncut segments before the linear amplification step in both the Bsu36I-treated and untreated samples.

In addition to the enrichment of G to T and C to T mutations at the ROI site, a G to T enriched mutation was also found at position 14 of HBB and HBD, two residues away from the Bsu36I site (FIG. 12). Indeed, 8-oxoG has been shown to affect neighboring bases and to compromise enzymatic digestion when placed near a restriction site. The finding that a complete G:C o T:A mutation (i.e., the mutation is fixed in both strands) at position 14 has no effect on Bsu36I digestion (FIG. 4) further supports the effect of a single-stranded change such as 8-oxoG on Bsu36I digestion.

Given their high frequencies and unbalanced distribution between the two strands that disqualify G to T, C to T and C to A substitutions in the target DNA strand as true, de novo mutations occurring in sperm cells, they were excluded (i.e., their sequencing output reciprocals C to A, G to A and G to T) from the calculation of mutation rates. By comparison, Jee et al. removed the mutations C to T, C to A and A to G from the MDS analysis of bacterial gene segments, suggesting that their high frequencies and strand occupancy bias reflect a snapshot image of base misincorporation and repair processes in the bacterial cells (Jee et al. Nature (2016) 534(7609):693).

### Example 9

### Repeated De Novo Mutations are not Due to Chimeric Duplication Events

As shown in Table 4, certain mutations were found to be enriched in the ROI of both HBB and HBD of the four donors by the Bsu36I treatment and are considered as true, de novo mutations. Of the overall 49 single-base substitutions that were found for all genes and donors, 14 occurred repeatedly in the same gene and in the same donor. Of the six enriched deletion mutations that were found, the Hb-Leiden mutation (a deletion of either codon 6 or codon 7, which results in the same sequence) occurred repeatedly in HBB in seven of the 11 samples and in HBD in two of the 11 samples. Methodological artifacts cannot explain the correspondence that is seen between de novo origination rates and observations of alleles in populations, as described Example 11. It was additionally confirmed independently that these repetitions are not due to duplications of mutant families by artifactual chimera that are generated during library preparation.

Chimeric sequences arising during PCR amplification are a common source of NGS sequencing artifacts, ranging from a few percent to nearly half of the sequences in individual libraries. A chimeric sequence can be generated during PCR due to low processivity of the DNA polymerase or insufficient elongation time that produce an incomplete DNA strand. Such a strand can anneal in one of the following cycles to a full-length strand of a second allele or a paralogue gene and complete its extension, thus creating an Allele1/Allele2, or a Gene1/Gene2 chimeric product in addition to the PCR products of the two alleles, or genes, respectively. Therefore, a similar mechanism involving the interaction between an incomplete strand of a mutation variant and a full-length strand of a wild-type variant could theoretically result in the duplication of the mutation variant. Specifically, if a mutation-carrying strand ends prematurely and loses its primary barcode, when serving as a mega primer in the following PCR cycle it will acquire a new barcode and could potentially lead to a second family that carries the mutation, where, in the unlikely event that such a family passes the combined filtration criteria, that second family is a false positive.

Since HBB/HBD chimeras in the present experiment are identifiable, as they carry both HBB and HBD-specific markers on different sides of the chimeric breakpoint (exemplified, for instance, by the relatively high frequency of HBB 9C to T or HBD 9T to C mutations), the HBB/HBD chimeras were used to estimate the probability that two separate families (each with its own primary barcode) that carry the same mutation actually arose from one family due to a chimeric event and thus represent a double-counting of the mutation. Specifically, since HBB and HBD share a high sequence identity, HBB/HBD chimeric artifacts could be generated as explained, by extending an incomplete strand of one paralog while using the full-length strand of the other paralog as a template during library preparation. Thus, the extended strand acquires the primary barcode of the template strand. As HBB and HBD reads are sorted into distinct sequence analysis pools based on their unique sequence markers, both the chimeric family and the “template” family were identified by their shared primary-barcode sequences and removed from further analysis (Example 6). For AFR1, AFR2 and AFR3, who exhibited multiple instances of the HbS mutation, about 1% or less of the Bsu36I-treated families that passed the combined filtration criteria were identified as potential HBB/HBD chimeras (Table 2). Since each chimeric event between a mutant strand and a wild-type strand can result in either a wild type or a mutant duplication, the fraction of observed mutant families that constitutes artifactual duplicates of other mutant families during PCR is at most half the fraction of chimerism, namely <0.5%. This per-mutant probability of artificial duplication is unable to account for the recurrence of mutations in the presented data, as not a single mutation is expected in the dataset to be a false positive due to double counting. Specifically, it is unable to explain the repetition of HbS and Hb-Leiden in the data.

### Example 10

### Genome-Wide Average Point Mutation, Indel and A to T Rates

The human genome-wide point mutation rate per base per generation is generally considered to be close to 1×10⁻⁸. Most recent estimates fall within the range 1-1.5×10⁻⁸. Thus, the midpoint of this range, 1.25×10⁻⁸, was used as a reference point for the sake of comparisons. Studies of the whole-exome mutation rate per base per generation average a bit higher, around 1.5×10⁻⁸. However, while many of these studies are based on individuals with a given disease, whole-exome mutation rates from healthy individuals or neutral sites have reported rates closer to the 1.25×10⁻⁸ reference point. Either way, whether using 1.25 or the relatively high 1.5 as a reference point, no significance assignment is affected. Furthermore, since the human genome-wide per base per generation indel rate is more than 10× smaller than the human genome-wide per base per generation point mutation rate, 1.25×10⁻⁹ was used as a slightly conservative reference point for the latter.

To obtain a per-base point mutation rate across an ROI that can be compared to previous measures of the genome-wide average per-base point mutation rate, the fact that 12 out of 18 of the possible point mutations across a single instance of the ROI are observable due to G to T, C to T and C to A exclusion, was taken into account. The effective average per-base point mutation rate observed, μ^{ROI}, is then obtained as follows:

$${µ}^{ROI} = \frac{M}{( N \times 12 / 3 )}$$

where M is the total number of point mutations observed across the ROI and N is the total number of families analyzed, namely the primary barcode families that have passed the combined cutoff criteria. In other words, the total number of point mutations observed is divided by the maximal number of point mutations that could have been observed, where the latter is divided by 3 to obtain a per-base rate that can be compared to previous measures of the genome-wide point mutation rate per-base, since 3 mutations are possible per base. This simple calculation is suitable for the purpose of testing whether the average point mutation rates observed across the ROIs are higher than previously measured genome-wide point mutation rates, because here the ROI rate is inferred from 9 out of 12 possible point mutation types, where the average rate of the excluded mutation types is expected a priori to be no lower than the average rate of the included ones based on previous knowledge of mutation rates per type.

The advantage of this method of comparing the ROI average to the genome-wide average per-base point mutation rate is that it takes into account the particular sequence at the ROI. Alternatively, taking the ROI per-base point mutation rate that is due to the 9 observable point mutation types only and comparing it to its genome-wide equivalent would require a complex adjustment of the genome-wide measure, whereas the goal here is merely to provide a general sense comparison to a well-known figure.

To obtain a per-base indel rate across an ROI, the total number of indel events (in the samples exemplified herein, only deletions have been observed) is divided by the total number of bases examined across primary barcode families by MEMDS. Here, a complication arises from the fact that not only indels that are entirely contained within the ROI can be observed but also indels that partly overlap with the ROI, as those too are captured and enriched by MEMDS. A simple way of addressing this fact is to expand the number of base positions examined to include all positions between the farthest upstream and downstream breakpoints observed in the dataset, namely between position 14 and 24 (a stretch of 11 positions). The denominator of the indel rate calculation is then the total number of families observed multiplied by 11. For testing whether the observed indel rate is higher than expected from the genome-wide average, this simple method is slightly conservative, because indels that overlap with the region between positions 14 and 24 but not with the ROI (e.g., a 12-14 deletion) are potentially possible but not observable and do not contribute toward the indel count.

To explain the calculation of the genome-wide average rates for the observable point-mutation types, the A to T transversion is taken for example. Based on a subset of de novo mutations with phasing information, the A:T to T:A transversion accounts for ˜6.5% of the total of point mutations across the human genome in males, while the A:T content across the human genome is ˜59%. Therefore, the average A to T mutation rate per adenine base per generation in males can be estimated as follows:

$$1.25 \times {10}^{- 8} \times \frac{6.5 \%}{59 \%} = 1.377 \times {10}^{- 9} < 1.4 \times {10}^{- 9} \qquad ( 6 )$$

Using a similar calculation with data on the relative frequencies of Extremely Rare Variants (ERVs) allows to obtain the A to T mutation rates in the 3-mer, 5-mer and 7-mer contexts relevant to the HBB and HBD ROIs, namely the GAG, TGAGG and CTGAGGA contexts, respectively (using the supporting materials of Carlson J, et al. (2018) Nat Commun. 9(1):3753), which are approximately 1.3, 1.2 and 0.9×10⁻⁹, respectively. In the same way, the genome-wide average rates, with or without the extended contexts, are obtained for the other observable point-mutations (Table 5). Thus, the local sequence up to the 7-mer context does not explain the high de novo origination rate of the A to T mutation in the HBB ROI in the African samples, consistent with the implication of complex genetic influences on the mutation-specific mutation origination rate. The same is true for the other point mutations that deviate from their expected genome-wide average rates (Table 5).

TABLE 5
Fold change of observed de novo rates from the genome-wide average (GWA) rates for point mutations in the African HBB ROI

| | | De novo | | | | | | | |
|---|---|---|---|---|---|---|---|---|---|
| | | mutation-based | | ERV-based, 3-mer | | ERV-based, 5-mer | | ERV-based, 7-mer | |
| Change description | | GWA rate | Fold | GWA rate | Fold | GWA rate | Fold | GWA rate | Fold |
| Mutation | Name (HbVar) | (×10⁻⁸) | change | (×10⁻⁸) | change | (×10⁻⁸) | change | (×10⁻⁸) | change |
| 16C > G | Hb Gorwihl | 0.21 | *7.4 | 0.27 | 5.8 | 0.28 | 5.7 | 0.36 | 4.4 |
| 16C > T | Hb Tyne | 0.73 | 2.2 | 0.78 | 2.0 | 0.97 | 1.6 | 0.79 | 2.0 |
| 17C > G | Hb Warwickshire | 0.21 | ***12.3 | 0.27 | ***9.7 | 0.17 | ***15.1 | 0.16 | ***16.4 |
| 17C > T | Hb Aix-les-Bains | 0.73 | 2.2 | 0.84 | 1.9 | 0.66 | 2.4 | 0.60 | 2.6 |
| 18T > G | | 0.17 | 3.1 | 0.17 | 3.1 | 0.16 | 3.3 | 0.16 | 3.2 |
| 18T > A | | 0.14 | 3.8 | 0.13 | 4.0 | 0.13 | 4.1 | 0.13 | 3.9 |
| 18T > C | | 0.59 | 1.8 | 0.55 | 1.9 | 0.59 | 1.8 | 0.76 | 1.4 |
| 20A > G | Hb Lavagna | 0.59 | **4.4 | 0.37 | **7.0 | 0.29 | ***9.1 | 0.28 | ***9.5 |
| 20A > T | HbS | 0.14 | ****34.4 | 0.13 | ****37.6 | 0.12 | ****40.4 | 0.09 | ****50.7 |
| 20A > C | Hb G-Makassar | 0.17 | 3.1 | 0.13 | 4.1 | 0.12 | 4.4 | 0.12 | 4.5 |
| 21G > C | | 0.21 | 2.5 | 0.27 | 1.9 | 0.22 | 2.4 | 0.18 | 3.0 |
| 22G > C | Hb Bellevue III | 0.21 | 0 | 0.27 | 0.0 | 0.28 | 0.0 | 0.20 | 0.0 |

*p = 0.0082
**p < 0.0060
***p < 3 × 10⁻⁴
****p < 10⁻¹⁰

Names of clinically known variants were taken from the Globin Gene Server database (Hardison RC, et al. (2002) Hum. Mutat. 19(3):225-233) GWA rates were calculated based on a subset of de novo mutations with phasing information taken from genome wide family sequencing studies (Rahbari R, et al. (2016) Nat Genet 48(2):126) as well as based on relative frequencies of Extremely Rare Variants (ERVs) for the 3-mer, 5-mer and 7-mer genetic contexts (Carlson J, et al. (2018) Nat Commun. 9(1):3753). The significance of the deviations of the mutation-specific origination rates observed here from the GWA rates, is not affected by taking into account the local genetic context in three out of four cases, in accord with the fact that adjustments to the GWA rates based on context are minor compared to the variation in mutation-specific origination rates observed here. Out of all the point mutation types studied here (twelve), the mutation-specific origination rate of the HbS mutation deviates by far the most from its GWA rate, even when taking into account the local genetic context. This effect is only strengthened in the larger contexts, as the origination rate of the HbS mutation deviates by ˜35× from its GWA calculated based on de novo mutation studies and by ˜38×, ˜40×, and ˜51× from its GWA calculated based on ERVs for the 3-mer, 5-mer and 7-mer contexts, respectively.

### Example 11

### The Correspondence Between De Novo Rates and Observations of Alleles in Populations

Twelve point-mutation types in each ROI are observable by the method of the invention, all of which have been observed to occur de novo in at least one ethnicity and ROI. In addition, 23 deletions of up to size 3 are observable in each ROI (considering that size 2 and 3 observable deletions reach beyond the boundaries of the ROI, and that size 1 deletions at position 19 are not observable).

The expected rate of indels decreases with indel size. Thus, because of the rarity of indels size >3, capping the analysis at this size provides for conservative P values, as including more indels would have only increased the fraction of indels that both have not been reported on Hb Var and that are not observed de novo. (Indeed, the next deletion reported on Hb Var that could have been observed by the method of the invention, 20_45del, is of size 26). Insertions are also relatively rare, and neither were reported before in the ROIs in Hb Var nor are observed here de novo, and thus their exclusion is also conservative for the statistical tests.

Of the point mutations, 8 have been reported on Hb Var (16 C to G, 16 C to T, 17 C to G, 17 C to T, 20 A to G, 20 A to T, 20 A to C, and 22 G to C), and of the 23 observable indels of up to size 3, 5 have been reported on Hb Var: 16delC, 17_18delCT, 18_19delTG, 19_21delGAG or the equivalent 22_24delGAG (the Hb-Leiden mutation), and 20delA, all in HBB (Table 5). Hb Var is an online database that gathers reports of all human hemoglobin variants from the literature and is arguably the largest source of information on this topic (Hardison RC, et al. (2002) Hum. Mutat. 19(3):225-233).

The de novo rates of deletion types reported on Hb Var are significantly higher than the rates of deletion types not reported on Hb Var, both combining the two ROIs (P=0:0033, two-sided permutation test) and for the HBB (P=0:029) and HBD ROIs separately (P=0:0056). However, because the Hb-Leiden mutation has an exceptionally high de novo rate among the deletions, in order to find out whether this mirroring between deletion de novo rates and reports of these mutations on Hb Var is driven by the Hb-Leiden mutation alone or extends also to other mutations, the fraction of deletion types reported on Hb Var that have been observed de novo at least once in the present data was compared to the same fraction among deletion types not reported on Hb Var. The former fraction is significantly larger than the latter both when combining the two ROIs (P=0:0078, two-sided Fisher exact test) and for each ROI separately (HBB: P=0:048, HBD: P=0:0056, two-sided Fisher exact test). While these tests already show that the results are not driven only by the high Hb-Leiden de novo rate, when combining the two ROIs due to the similarity of indel types observed between them, the results remain significant also when excluding the Hb-Leiden mutation completely (P=0:026 and P=0:024, two-sided Fisher exact test), further demonstrating that the mirroring between these mutation's de novo rates and observations of them in populations extends beyond the Hb-Leiden mutation. Indeed, note that of the 6 indel types that were observed de novo in any ROI and ethnicity, 4 are included in the 5 deletions reported on Hb Var (16delC, 17_18delCT, 19_21delGAG or 22_24delGAG, and 20delA), and of the deletions reported on Hb Var, only one has not been observed de novo (18_19delTG). In accord with this observation, most of the deletion mutations that were observed can already be seen to be taking part in the mirroring effect mentioned above.

For the point mutations, a much smaller list of observable types is available, all of which have been observed de novo at least once. In addition, 3 out of the 4 point-mutations not reported on Hb Var are synonymous (18T to G, 18T to A and 18T to C), for which reason one could not have expected them to be reported on Hb Var to begin with. Therefore, it is not possible to apply the mirroring analysis above to the point mutations. Examining instead the frequencies of alleles in the ROIs reported in population genetic data from gnomAD exome sequencing provided by Ensembl (release 102) (Yates A, et al (2020) Ensembl 2020. Nucleic Acids Research 48: D682-D688), where only sparse data is available, we find that only 3 point-mutations in total were reported at non-zero frequencies, all in HBB. These are the HbS mutation, 16C to G and 16C to T. Two are of high and one is of middle de novo rate in the presented data. It is also found that the Hb-Leiden mutation, notably the most frequent de novo mutation also in the HBD ROI, is the only variant of non-zero frequency reported on gnomAD in the HBD ROI.

To conclude, the correspondence between de novo rates and observations of alleles in populations applies to both types of mutation and extends beyond the HbS and Hb-Leiden mutations. Further adding to this analysis, while HbS is common mostly in Africa and in some populations in the Asian malaria belt, it appeared de novo in the African but not in the European donors, and while the Hb-Leiden mutation has been reported across the globe, it appeared de novo in both the African and European donors.

The correspondence above-mentioned could not have been predicted from the genome-wide average (GWA) rates of the mutations involved. In particular, different indel GWA rates are not assigned to different indel types as defined here but to different indel sizes (the smaller the more frequent), a minor effect which only further contrasts with the fact that the Hb-Leiden deletion (the largest shown here) has the highest de novo rate. In addition, the HbS mutation is an A to T transversion, the least frequent point mutation type on average.

### Materials and Methods

All the oligos for the sperm DNA library preparation were ordered from IDT with standard desalting purity, unless otherwise mentioned. All enzymes were obtained from New England Biolabs (NEB). PCR purification and agarose gel extraction were carried out with QIAGEN kits.

### Preventing Cross-Contamination Between Samples

Because of the ultra-high accuracy of the method, preventing cross-contamination between samples is of the utmost importance. DNA was extracted from semen and prepared for next generation sequencing one donor at a time. Ultra-pure water (DNase and RNase-free) was used for all applications, from semen DNA extraction to library preparation. When possible, reagents were purchased in ready-to-use solutions and used in aliquots that were disposed after each library-preparation cycle together with other dry disposable materials. Non-disposable instruments were cleaned and when possible sterilized according to the manufacturer's instruction.
Working surfaces were cleaned by labZAPTM (A&A Biotechnology) to remove traces of nucleic acid, and the molecular biology work was confined to UV-sterilized cabinets. A spatial separation between pre- and post-PCR procedures was maintained by performing the pre-PCR steps in one lab and the post-PCR steps in a second, remote lab. A unidirectional flow of materials was maintained so that samples, reagents or other items were allowed to move from the pre-PCR lab to the post-PCR lab but never in the opposite direction. Plasmids were constructed in a separate room, mixed, and diluted to a working concentration and only then transferred to the pre-PCR room. Other than this final plasmid mix, no reagents or other materials were moved from the plasmid room to the pre-PCR lab area. Additionally, each library was generated with two unique identifier sequences that were added during the primary and the secondary barcode labeling steps, so that if any amplicon sequence from a previous analysis unrelated to the present analysis has infiltrated the sample during one of the library preparation steps, it could be eliminated during the sequence analysis step.

### Spike-In Plasmids Preparation

Four puc 19-based plasmids were generated. Two (ALP13 and ALP17) were designed to carry the HBB genomic segment from position −203 to +223 relative to the mRNA translation start site, with the Bsu36I restriction site CCTGAGG replaced with TTATGTT and ACGAGAC, respectively; and two others (ALP16 and ALP18) were designed to carry the HBD genomic segment from position −59 to +220 relative to the mRNA translation start site, with the Bsu36I restriction site replaced with TTATGTT and ACGAGAC, respectively. To prepare the spike-in mixture, the four plasmids were linearized by BamHI, mixed in equal amounts and diluted to 10 femtograms/μl for the AFR1, AFR3, AFR5, AFR6, AFR7, EUR3 and EUR4 samples and 5 femtograms/μl for all other samples.

### Collection of Sperm Samples

Semen samples from Africans were collected in the Assisted Conception Unit of the Lister Hospital & Fertility Centre in Accra, Ghana following clinical standards, and semen samples from Europeans were purchased from Fairfax, a large US cryobank, with the approvals of the Institutional Review Board of the Noguchi Memorial Institute for Medical Research (NMIMR-IRB 081/16-17) at the University of Ghana, Legon, and of the Rambam Health Care Center Helsinki Committee (0312-16-RMB) and the Israel Ministry of Health (20188768). Donors with a history of cancer or infertility or with high fever in the last 3 months prior to donation were excluded. Informed consent was obtained from all participants and personal identifying information was removed and replaced with codes at the source. The samples were shipped in dry ice or liquid nitrogen according to availability to the University of Haifa for analysis.

### DNA Extraction from Sperm Cells

The DNA isolation protocol is a modified version of the method described by Weyrich (Curr. Protoc. Mol Biol 98(1):2-13 (2012)). A semen sample from a single donor was divided into 500 μl aliquots in multiple screw-capped tubes. The sperm aliquots were washed twice with 70% ethanol to remove seminal plasma. The remaining cells were rotated overnight at 50° C. in a 700 μl lysis buffer (50 mM Tris-HCl pH 8.0, 100 mM NaCl₂, 50 mM EDTA, 1% SDS) containing 0.5% Triton X-100 (Fisher BioReagents BP151-100), 50 mM Tris(2-carboxyethyl) phosphine hydrochloride (TCEP; Sigma 646547) and 1.75 mg/mL Proteinase K (Fisher BioReagents BP1700-100). Lysates were centrifuged at 21,000×g for 10 minutes at room temperature and supernatants were united in a single tube. DNA purification from the cleared lysate was carried out using QIAGEN Blood & Cell Culture DNA Maxi Kit (13362). Specifically, 5 mL lysate were supplemented by 15 mL of buffer G2 (800 mM guanidine hydrochloride, 30 mM Tris-HCl pH 8.0, 30 mM EDTA pH 8.0, 5% Tween 20, 0.5% Triton X-100), vortexed thoroughly and allowed to gravity-flow through a single Genomic-tip 500/G column pre-equilibrated by 10 mL of buffer QBT (750 mM NaCl, 50 mM MOPS pH 7.0, 15% isopropanol (v/v)). Resin was washed twice by 15 mL of Buffer QC (1 MNaCl, 50 mMMOPS pH 7.0, 15% isopropanol (v/v)) and elution was carried out by 15 mL of Buffer QF pre-warmed to 50° C. (1.25 M NaCl, 50 mM Tris-HCl pH 7.0, 15% isopropanol (v/v)). DNA was precipitated by adding 10.5 ml room temperature isopropanol to the elute, inverting the tube 10 times, and using a sterile tip to spool and transfer the DNA to a screw capped tube containing 500 μl of buffer EB (10 mM Tris-HCl pH 8.5). The DNA dissolved overnight at room-temperature. For each donor, a small aliquot from the extracted DNA was PCR amplified and Sanger sequenced to verify the exact sequence of HBB and HBD regions.

### Enzymatic Digestion

For the RE-1-treated sample, roughly 264 μg sperm DNA, equivalent to 80 million haploid cells (For AFR2 DNA amount equivalent to 60 million cells was used), were mixed with a plasmid spike-in mixture (0.2 pg for AFRI and 0.1 pg for other donors) and equally divided in a 96-well plate. Bsu36I-HF digestion (RE-1) was carried out overnight at 37° C. according to the manufacturer's instructions using 5 units per well. Then, each well was supplemented by 6 units of HpyCH4III and digestion continued for three more hours. For the RE-1-untreated reaction (no Bsu36I digest), 13.2 μg sperm DNA (and 9.9 μg for AFR2), representing 5% of the DNA amount used for Bsu36I digest, were mixed with 6 times the volume of plasmid spike-in mixture, aliquoted to 5 tubes and incubated overnight with 2 units Sall-HF per tube instead of Bsu36I to allow for similar conditions of DNA digestion without affecting the Bsu36I and HpyCH4III sites. Then, each well was supplemented by 6 units of HpyCH4III (RE-2) and digestion continued for three more hours. All digestion products were purified using a QIAGEN PCR purification kit.

### Primary Barcode Labeling and Linear Amplification

Direct barcode labeling and linear amplification of the digested HBB and HBD strands were carried out in a single reaction in 96-well plates. Each well contained about 1 μg of digested DNA, 0.1 μM primary-barcode oligo (oligo A) and 1 μM of 5′-phosphorothioate-protected primer for linear amplification (oligo B). The reaction was carried out with Q5 high-fidelity polymerase according to the manufacturer's instructions, using the following thermocycler parameters: initial denaturation at 98° C. for 20 seconds, followed by 16 cycles of 98° C. for 5 seconds, 68° C. for 15 seconds, and 72° C. for 20 seconds. The DNA was purified using a PCR purification kit. For each donor, each of the Bsu36I-treated and untreated samples was labeled by an oligo A with a different Donor identifier-1 (ID-1) sequence, which was also not shared by samples from other donors, making it so that each donor and each condition had its own identifier sequence.

### 5′-Exonuclease Treatment

To eliminate non 5′-phosphorothioate-protected strands, 15 μg DNA aliquots from the post linearly amplified product of the Bsu36I-treated sample were incubated each at 37° C. in the presence of 15 units of Lambda exonuclease, 30 units of T7 exonuclease and 90 units of RecJF exonuclease in 1× CutSmart buffer for 2.5 hours. The post linearly amplified product of the Bsu36I-untreated sample was incubated at the same conditions with 10 units of Lambda exonuclease, 20 units of T7 exonuclease and 60 units of RecJF exonuclease. The DNA was purified using a QIAGEN PCR purification kit.

### Secondary-Barcode Labeling and 3′-Exonuclease Treatment

The DNA was aliquoted into a 96-well plate (1 μg per well). A single primer extension reaction was carried out using 0.5 μM of the secondary-barcode primer (oligo C) and Q5 high-fidelity polymerase according to manufacturer's instructions. The following thermocycler parameters were used: initial denaturation at 98° C. for 20 seconds, followed by a single cycle of 98° C. for 5 seconds, 68° C. for 15 seconds, and 72° C. for 40 seconds. To remove excess oligo C, immediately after the thermocycler temperature dropped to 16° C., 20 units of thermolabile Exo I were added directly to each well together with the relabeling control primer (oligo D) in a known amount equivalent to 0.66% of the secondary-barcode primer. After incubation of one hour at 37° C., the thermolabile Exo I was heat-inactivated by one minute at 80° C. and the DNA was purified using a PCR purification kit. For each donor, each of the Bsu36I-treated and untreated samples was labeled by an oligo C with a different Donor identifier-2 sequence (ID-2), which was also not shared by samples from other donors, making it so that each donor and each condition had its own identifier-2 sequence.

### PCR Amplification and Sequencing

The first PCR reaction of the dual barcode labeled product was carried out using oligo E and oligo F1 as primers and Q5 high-fidelity polymerase, according to manufacturer's instructions. The following thermocycler parameters were used: initial denaturation at 98° C. for 30 seconds, followed by 10 cycles of 98° C. for 5 seconds, 72° C. for 15 seconds, 72° C. for 30 seconds, and a final extension at 72° C. for 30 seconds. Amplification products were purified using a PCR purification kit. The second PCR reaction was carried out using 25% of the first PCR product as template, the amplification primers E and F2, and Q5 high-fidelity polymerase according to the manufacturer's instructions (different F2 primers were used to add a unique Illumina index sequence to each Bsu36I-treated and untreated sample). The following thermocycler parameters were used: initial denaturation at 98° C. for 30 seconds, followed by 24 cycles (except for EUR4 sample that was amplified by 17 cycles) of 98° C. for 5 seconds, 70° C. for 15 seconds, 72° C. for 30 seconds, and a final extension at 72° C. for 1 minute. PCR products were agarose-gel purified using QIAGEN gel extraction kit, and further concentrated by a DNA clean & concentrator kit (Zymo Research). DNA libraries prepared from the Bsu36I-treated and untreated samples of the same donor were mixed in equal amounts and paired-end sequenced with 20% PhiX by Illumina MiSeq 300 cycles kit (V2) at the Technion Genome Center (TGC). For each donor two or three MiSeq runs were performed to reach a minimum of 10 million reads per treatment (specifically, all but AFR5 and EUR3 were sequenced two times) and the resulting fastq sequences were joined prior to the sequence analysis step.

### Sequence Analysis

Illumina paired end (PE) reads were merged via Pear using the default model for the detection of significantly aligned regions and Phred score corrections. Merged sequences were trimmed from Illumina adapters using Cutadapt, and quality filtered by Trimmomatic using a sliding window size of 3 and a Phred quality threshold of 30. Quality filtered sequences were trimmed to remove the 5′ edge up to position 18, a sequence which includes the 14 bases of the primary barcode and the 4 bases of ID-1, while adding this information to the read's header. Only sequences with the correct ID-1 and first three bases of HBB or HBD sequences were maintained. Similarly, sequences were trimmed from 9 bp at their 3′ edge, which include the 5 bases of the secondary barcode and the 4 bases of ID-2, while adding this information to the read's header. Only sequences with the correct ID-2 were maintained. Trimmed sequences were sorted to HBB or HBD sequence pools, based on the occupying bases at positions 33-38 of the coding sequence (CGTTAC for HBB and TGTCAA for HBD), allowing one mismatch and frameshifts of up to −3 or +3. Successfully sorted sequences were mapped to either the HBB or HBD reference sequences (obtained by sanger sequencing aliquots from the matching donor samples) using BWA (parameters −M −t), and high-quality mutations (Phred score≥28) were noted. Reads were grouped by their primary barcodes to ‘families’ and processed according to the workflow depicted in FIG. 8.

### Bsu36I Single-Base Substitution Sensitivity Assay

Nineteen oligos (BSU 1-19) carrying the first 37 bases of HBB, each with a randomized nucleotide at a single position within the seven bases of the Bsu36I recognition site or at one of the six bases that flank this site from either side were mixed with a similar oligo with all the seven bases of the Bsu36I recognition site replaced by the sequence TTATGTT (Bsu36IR). This oligos mixture was PCR-amplified for 25 cycles using Q5 DNA polymerase and primers that match the Illumina adapter sequences flanking the HBB region in each oligo (Primers BSU F1 and BSU R1). 150 ng of this PCR product were subjected to 20 hours incubation at 37° ° C. with or without 5 units Bsu36I. Digestion products were purified, re-amplified (Primers BSU F2 and BSU R2) and paired end sequenced by Illumina MiSeq. To calculate Bsu36I sensitivity, mutation variant reads were counted and the frequency of each variant in the Bsu36I-treated sample was divided by its frequency in the Bsu36I-untreated sample. All ratios were normalized to the ratio of the Bsu36IR variant that was considered 100% resistant to Bsu36I.

### DNA Oligos

### Oligos for Sperm DNA Library Preparation

### Name: Oligo A

Description: Direct attachment of primary barcode

Sequence:
(SEQ ID NO: 1)
P-
CTCTTTCCCTACACGACGCTCTTCCGATCT(14N)RRRRWGTGTTCACT
AGCAAYCCTCAAACAGACACC-InvdT

Sequence features:
P-5′ phosphate, to improve subsequent degradation by 5′ exonucleases. In italics—part of Illumina TruSeq Universal Adapter P5 that carries the sequence for read-1 sequencing primer. (14N)—Primary Barcode, unique to each labeled molecule. RRRR—Donor identifier (ID)-1, a sequence of four bases unique to each donor. Underlined sequence—complementary to HBB and HBD antisense strands; covers 30 bases between HpyCH4III digestion site and the minus 1 position relative to the mRNA translation start site; W (either A or T) was designed to equally prefer the single base difference between the 3′ terminus of HBB (3′T) and HBD (3′A) antisense strands produced by HpyCH4III digestion. Y—a base insertion designed to identify events of erroneous extension and amplification promoted by unblocked (3′ InvdT missing) Oligo A, if any. InvdT—3′ inverted dT modification, designed to block extension by Q5 DNA polymerase.

### Name: Oligo B

Description: Linear amplification of barcoded strand

Sequence:
(SEQ ID NO: 2)
ApsApsTpsGpsApsTACGGCGACCACCGAGATCTACACTCTTTCCCTA
CACGACGCTC

Sequence features:
Underlined sequence—region complementary to the fill-in product (barcode-labeled strand) by Oligo A. In italics—completes the 5′ part of Illumina TruSeq Universal Adapter P5. ps—phosphorothioate; protect linearly amplified sequences from 5′ exonuclease degradation.

### Name: Oligo C

Description: Secondary barcode

Sequence:
(SEQ ID NO: 3)
GTGACTGGAGTTCAGACGTGTGCTCTTCCGATCT(5N)RRRRATCCACG
TTCACCTTGCYCCACAGGGCAGTAAC

Sequence features:
In italics—Part of Illumina TruSeq Universal Adapter P7 that carries the sequence for read-2 and the index sequencing primers. (5N)—Secondary Barcode, unique to each linearly amplified sequence. RRRR—Donor identifier (ID)-2, four bases unique to each donor; Underlined sequence—region complementary to HBB and HBD sequence. Y—perfect match for HBB, a mismatch for HBD.

### Name: Oligo D

Description: Relabeling control primer

(SEQ ID NO: 4)
Sequence:
GTGACTGGAGTTCAGACGTGTGCTCTTCCGATCTAGTGTAAAAATCCAC
GTTCACCTTGCYCCACAGGGCAGTAAC

Sequence features:
Identical to Oligo C, but with the sequence AGTGT replacing the Secondary barcode sequence (5N), and AAAA replacing ID-2 sequence RRRR (underlined).

### Name: Oligo E

Description: Forward amplification primer for first and second PCR reactions

Sequence:
(SEQ ID NO: 5)
AATGATACGGCGACCACCGAGATCTAC

Sequence features:
Matches the 5′ edge generated by Oligo B

### Name: Oligo F1

Description: Reverse amplification primer for first PCR reaction

Sequence:
(SEQ ID NO: 6)
GTGACTGGAGTTCAGACGTGTGCTC

Sequence features:
Matches the 3′ edge generated by Oligo C

### Name: Oligo F2

Description: Reverse amplification primer for second PCR reaction

Sequence:
(SEQ ID NO: 7)
CAAGCAGAAGACGGCATACGAGATRRRRRRGTGACTGGAGTTCAGACGTG
TGC

Sequence features:
Completes the Illumina TruSeq Universal Adapter P7; RRRRRR stands for the Illumina index sequence.

### Oligos for Bsu36I-Mutation Sensitivity Test

### Name: BSU 1-19

Description: Template oligos for single-base mutation library generation by PCR

Sequence:
(SEQ ID NO: 8)
TACACGACGCTCTTCCGATCTATGGTGCAYCTGACTcctgaggAGAAGTC
TGCCGTTAAGATCGGAAGAGCACACGTCTG

Sequence features:
In Italics—part of Illumina TruSeq Universal Adapters P5 and P7. In bold—the first 37 bases of HBB WT sequence beginning from the mRNA translation start site (ATG). In lowercased letters—Bsu36I recognition site. Underlined sequence—19 bases in HBB sequence that were subjected to randomized mutagenesis. Each oligo from BSU1 to BSU19 carries a randomized base mixture at the equivalent position (N, N, N, B, D, V, D, D, V, H, B, H, H, B, H, B, N, N, N, respectively).

### Name: BSU 20 (Bsu36I^{R})

Description: Bsu36I resistance variant

Sequence:
(SEQ ID NO: 9)
TACACGACGCTCTTCCGATCTATGGTGCAYCTGACTttatgttAGAAGTC
TGCCGTTAAGATCGGAAGAGCACACGTCTG

Sequence features:
Identical to the Oligo described for BSU 1-19, but with the Bsu36I recognition sequence replaced from cctgagg to ttatgtt.

### Name: BSU F1

Description: Forward primer for generating double stranded BSU library (dsBSU) by PCR together with BSU R1 primer and BSU1-20 oligos' mixture as a template.

Sequence:
(SEQ ID NO: 10)
ACACTCTTTCCCTACACGACGCTCTTCCGATCTATGGTGCA

Sequence features:
In Italics - part of Illumina TruSeq Universal Adapters P5. In bold—5′ of HBB segment. Underlined—sequence matching BSU 1-20 oligos.

### Name: BSU R1

Description: Reverse primer for generating double stranded BSU library (dsBSU) by PCR together with BSU F1 primer and BSU1-20 oligos' mixture as a template.

Sequence:
(SEQ ID NO: 11)
GTGACTGGAGTTCAGACGTGTGCTCTTCCGATCTTAACGGCA

Sequence features:
In Italics—part of Illumina TruSeq Universal Adapters P7. In bold—3′ of HBB segment. Underlined - sequence matching to BSU 1-20 oligos.

### Name: BSU F2

Description: Forward primer for generating the BSU library by PCR together with BSU R2 primer and Bsu36I treated and Nontreated dsBSU as templates, for next-generation sequencing.

Sequence:
(SEQ ID NO: 12)
AATGATACGGCGACCACCGAGATCTACACTCTTTCCCTACACGACGCTC
TTCCGATCTATGGTGCA

Sequence features:
Completes the Illumina TruSeq Universal Adapter P5. Underlined—sequence matching to dsBSU library.

### Name: BSU R2

Description: Reverse primer for generating the BSU library by PCR together with BSU F2 primer and Bsu36I treated and Nontreated dsBSU as templates, for next-generation sequencing.

Sequence:
(SEQ ID NO: 13)
CAAGCAGAAGACGGCATACGAGATRRRRRRGTGACTGGAGTTCAGACGT
GTGC

Sequence features:
Completes the Illumina TruSeq Universal Adapter P7. Underlined—sequence matching to dsBSU library.

1. A method of identifying genetic variants in DNA; said method comprising: a. Providing a sample of isolated DNA, wherein said DNA comprises wild-type DNA sequences and optionally one or more DNA sequences containing a genetic variant in one or more regions of interest (ROIs); b. Removing said wild-type DNA sequences from said sample, thereby enriching the sample with DNA sequences containing the genetic variant; c. Determining the number of the wild-type sequences that were removed by calculating an enrichment factor (£); and d. Determining the number of genetic variants in said DNA sample.

2. The method of claim 1, wherein said step (b) of removing said wild-type DNA sequences from said sample comprises subjecting said DNA to a first cutting agent and optionally to a second cutting agent, wherein the recognition site for said first cutting agent is within the ROI, and wherein the recognition site for said second cutting agent is in proximity to the ROI, whereby wildtype ROI is cut by the first cutting agent and an ROI which comprises a genetic variation is not cut by said first cutting agent.

3. The method of claim 1, wherein said step (d) of determining the number of genetic variants in said DNA is performed by a sequencing-based method.

4. The method of claim 3, wherein said sequencing-method is a barcoding-based sequencing method.

5. The method of claim 4, wherein said barcoding-based sequencing method comprises: a. Attaching a Primary barcode to said ROI products thereby obtaining barcoded ROI products; b. Linearly amplifying said barcoded ROI products thereby obtaining amplified barcoded ROI products; c. Performing a polymerase chain reaction (PCR) with the amplified barcoded ROI product of step (ii) using at least two primers for next generation sequencing; and d. Sequencing the amplified product, thereby obtaining sequencing data; and e. Analyzing the data obtained in step (iv) to determine the number of genetic variants in said DNA.

6. The method of claim 1, wherein said step (d) of determining the number of genetic variants in said DNA is performed by a polymerase chain reaction (PCR).

7. The method of claim 6 wherein said PCR is quantitative PCR or digital droplet PCR, optionally wherein said genetic variants are ultra-rare genetic variants, and optionally wherein: (i) said genetic variants are one or more de novo mutations; (ii) said one or more de novo mutation is a specific predefined de novo mutation; or both (i) and (ii).

8.-10. (canceled)

11. The method of any one of the preceding claim 1, wherein any one of: (i) said sample comprises a cell population comprising between about <10,000 cells and about 1×10⁹ cells; (ii) said method identifies genetic variants with a maximal error rate of 1 per 400 million bases; (iii) said step (b) of claim 1 results in obtaining a population of isolated ROI products comprising one or more target mutations, wherein wild-type ROI sequences were substantially removed from said population; and (vi) any combination of (i) to (iii).

12.-13. (canceled)

14. The method claim 5, wherein any one of: (i) step (a) of claim 5 comprises forming a mixture comprising the cut DNA and an oligonucleotide, wherein said oligonucleotide comprises a primary barcode, a primer sequence, and optionally a sample-identifier sequence, and wherein said oligonucleotide anneals to the sequence between the recognition site of the first cutting agent and the recognition site of the second cutting agent, and optionally wherein said primer sequence is an Illumina P5-primer sequence; (ii) step (a) of claim 5 further comprises attaching a base modification that blocks DNA polymerase from extending said oligonucleotide and optionally further comprising planting a control single-base insertion, and optionally wherein said base modification that blocks DNA polymerase from extending said oligonucleotide is a 3′ inverted-dT (3′inv-dT); (iii) step (b) of claim 5 comprises performing one or more cycles of linear amplification using an oligonucleotide that anneals to the primer sequence of the target DNA strand, thereby obtaining copies in an amount that is equal or less than the number of linear cycles of each barcoded target molecule, and optionally wherein step (b) of claim 5 comprises performing between 2 cycles and 20 cycles of linear amplification; and (vi) any combination of (i) to (iii).

15.-19. (canceled)

20. The method of claim 5, wherein any one of: (i) the method further comprises subjecting said amplified barcoded ROI products obtained in step (b) of claim 5 to degradation by a 5′-exonuclease enzyme, optionally wherein the DNA is larger than 10 million base pairs, and optionally wherein said method further comprises adding an adapter sequence comprising one or more base modifications that protect a barcoded-ROI copy from 5′ exonuclease degradation at the 5′ edge (5′PS) of each barcoded-ROI copy.

21.-22. (canceled)

23. The method of claim 20, wherein any one of: (i) said adapter sequence is an Illumina adapter sequence or said adapter sequence comprises 5 base modifications; (ii) said base modifications are phosphorothioate bonds; (iii) the method further comprises attaching a secondary barcode after said amplified barcoded ROI products were subjected to degradation by a 5′-exonuclease enzyme, thereby obtaining a double-stranded barcoded ROI; and (vi) any combination of (i) to (iii).

24.-26. (canceled)

27. The method of claim 5, wherein: (i) the method further comprises degrading said amplified barcoded ROI using a 3′-exonuclease prior to sequencing the amplified product; (ii) said step of determining the number of the target wild-type sequences that were removed by calculating an enrichment factor (E) comprises the steps of: a. Providing mock DNA comprising copies of an artificial sequence that is resistant (R) to cutting by the first cutting agent;
b. Subjecting a fraction of said mock DNA to a first cutting agent and optionally to a second cutting agent, wherein the recognition site for said first cutting agent is within the ROI, and wherein the recognition site for said second cutting agent is in proximity to the ROI, whereby wildtype ROI is cut by the first cutting agent and an ROI which comprises a genetic variation is not cut by said first cutting agent; and further subjecting the fraction of said mock DNA to steps (a) to (d) of claim 5, thereby obtaining sequence data for mock DNA subjected to cutting (referred to herein as Group III); c. In parallel, subjecting another fraction of said mock DNA only to said second cutting agent; and further subjecting the fraction of said mock DNA to steps (b) to (d) of claim 5, thereby obtaining sequence data for mock DNA that was not subjected to said first cutting agent (referred to herein as Group IV); d. Providing isolated DNA comprising a region of interest (ROI) in accordance with claim 1 step (a); e. Subjecting a fraction of said DNA to a first cutting agent and optionally to a second cutting agent, wherein the recognition site for said first cutting agent is within the ROI, and wherein the recognition site for said second cutting agent is in proximity to the ROI, whereby wildtype ROI is cut by the first cutting agent and an ROI which comprises a genetic variation is not cut by said first cutting agent; and further subjecting the fraction of said DNA to steps (a) to (d) of claim 5, thereby obtaining sequence data for DNA subjected to cutting (referred to herein as Group I); f. In parallel, subjecting another fraction of said DNA only to said second cutting agent; and further subjecting the fraction of said DNA to steps (b) to (d) of claim 5, thereby obtaining sequence data for DNA that was not subjected to said first cutting agent (referred to herein as Group II); and g. comparing the ratios between the numbers of DNA molecules that are sensitive (S) and resistant (R) to the cutting agent between the mixture that was subjected to cutting the wild-type and the mixture that was not subjected to cutting the wild-type, thereby determining the number of the target wild-type sequences that were removed, and optionally wherein wherein the enrichment factor (E) is determined by the formula
$$E = \frac{Rfe \times Sfc \times VSe \times VRc}{Sfe \times Rfc \times VRe \times VSc}$$
wherein R^{f}e is the number of artificial, resistant molecules measured in Group III,
S^{f}c is the number of sensitive molecules measured in Group II;
Vsₑ is the volume taken from the DNA tube for Group I;
V_{RC} is the volume taken from the mock DNA tube for Group IV;
Sfₑ is the number of sensitive molecules measured in Group I;
R^{f}_{c} is the number of artificial, resistant molecules measured in Group IV;
V_{Re} is the volume taken from the mock DNA tube for treatment in Group III; and
Vsₑ is the volume taken from the DNA tube for Group II; or both (i) and (ii).

28. The method of claim 1, wherein step (c) of claim 1 is performed in parallel with step (b).

29.-30. (canceled)

31. The method of claim 5, wherein the step of analyzing the data obtained to determine the number of genetic variants in said DNA comprises using combined threshold criteria, wherein said criteria comprise: a. primary-barcode family size, b. within-family mutation frequency cutoff, and c. association of at least two secondary barcodes with each base, and optionally wherein said combined threshold criteria comprise: a. primary-barcode families with at least three reads, b. a minimal within-family mutation frequency cutoff of 70%, and c. the association of at least two secondary barcodes with each base.

32. (canceled)

33. The method of claim 5, wherein the step of analyzing the data obtained to determine the number of genetic variants in said DNA comprises the algorithm as shown in FIG. 8.

34. The method of claim 2, wherein said first cutting agent and said second cutting agent is an enzyme, and optionally wherein: said enzyme is an endonuclease or a restriction enzyme, said first cutting agent is an enzyme that cleaves at the ROI to produce digested DNA, or both.

35.-36. (canceled)

37. The method of claim 2, wherein: said first cutting agent and said second cutting agent are the same, said first and/or second cutting agent is a CRISPR-Cas9 agent, or both.

38.-39. (canceled)

40. The method of claim 1, wherein said sample of isolated DNA is a biological sample and wherein said biological sample is selected from a group consisting of semen, amniotic fluid, blood, cerebrospinal fluid, ascitic fluid, saliva, urine, bronchoalveolar lavage fluid, or nasal lavage fluid, or a tissue biopsy or wherein said sample of isolated DNA is a non-biological sample and wherein said non-biological sample is selected from a group consisting of a soil sample, a sewer sample, water sample, and a sample taken from a solid surface.

41.-48. (canceled)
